SEQUENCE LISTING (155 sequences)

SEQ ID NO: 1 - DNA, 10 bp, Artificial
  Linker for ligation of ChIPed DNA
acctcggccg

SEQ ID NO: 2 - DNA, 42 bp, Artificial
  Linker for ligation of ChIPed DNA
ctaatacgactcactatagggcagcgtggtcgcggccgaggt

SEQ ID NO: 3 - DNA, 20 bp, Artificial
  PCR primer for amplification of ChIPed DNA
agcgtggtcgcggccgaggt

SEQ ID NO: 4 - DNA, 20 bp, Artificial
  ChIP primer
ttaccaccgctctcatttca

SEQ ID NO: 5 - DNA, 20 bp, Artificial
  ChIP primer
ctgctgaaaagggcaagaag

SEQ ID NO: 6 - DNA, 20 bp, Artificial
  ChIP primer
tcagtgaaatcgctgctgtc

SEQ ID NO: 7 - DNA, 20 bp, Artificial
  ChIP primer
tggaggattcggatgacttc

SEQ ID NO: 8 - DNA, 20 bp, Artificial
  ChIP primer
agaagggtcaacacgtccac

SEQ ID NO: 9 - DNA, 20 bp, Artificial
  ChIP primer
tcgtgatggctgattctctg

SEQ ID NO: 10 - DNA, 20 bp, Artificial
  ChIP primer
caggcatactttgccctctc

SEQ ID NO: 11 - DNA, 20 bp, Artificial
  ChIP primer
tgactctgtgacccctaccc

SEQ ID NO: 12 - DNA, 20 bp, Artificial
  ChIP primer
cacagctttccttcctctgg

SEQ ID NO: 13 - DNA, 20 bp, Artificial
  ChIP primer
gggagacaaccccctacttc

SEQ ID NO: 14 - DNA, 20 bp, Artificial
  ChIP primer
aggaagtcacgaaagccaga

SEQ ID NO: 15 - DNA, 20 bp, Artificial
  ChIP primer
ggatggaaaagaggtcgtga

SEQ ID NO: 16 - DNA, 20 bp, Artificial
  ChIP primer
accgcaggatgaaactgaag

SEQ ID NO: 17 - DNA, 20 bp, Artificial
  ChIP primer
ttcccccagaaaacaacaaa

SEQ ID NO: 18 - DNA, 20 bp, Artificial
  ChIP primer
aagcaggagtcagtgggcta

SEQ ID NO: 19 - DNA, 20 bp, Artificial
  ChIP primer
gatgctggcctctggagtta

SEQ ID NO: 20 - DNA, 20 bp, Artificial
  RT-PCR primer
tgttcccacattccagatga

SEQ ID NO: 21 - DNA, 20 bp, Artificial
  RT-PCR primer
tcctccctctgcacaagact

SEQ ID NO: 22 - DNA, 20 bp, Artificial
  RT-PCR primer
ccagcaaaacttcaggggta

SEQ ID NO: 23 - DNA, 20 bp, Artificial
  RT-PCR primer
caaatcagaagtgccgtcct

SEQ ID NO: 24 - DNA, 20 bp, Artificial
  RT-PCR primer
agtgcatcggtatgcatgag

SEQ ID NO: 25 - DNA, 20 bp, Artificial
  RT-PCR primer
tgccatgcatcaaaataacc

SEQ ID NO: 26 - DNA, 20 bp, Artificial
  RT-PCR primer
gatgttggggcacaagaact

SEQ ID NO: 27 - DNA, 20 bp, Artificial
  RT-PCR primer
tcctgggagaagctcagtgt

SEQ ID NO: 28 - DNA, 20 bp, Artificial
  RT-PCR primer
tcattggctgcaagacagac

SEQ ID NO: 29 - DNA, 20 bp, Artificial
  RT-PCR primer
agcttttggccttttccttc

SEQ ID NO: 30 - DNA, 20 bp, Artificial
  RT-PCR primer
tattttgcgagaacccatcc

SEQ ID NO: 31 - DNA, 20 bp, Artificial
  RT-PCR primer
actgctgtctttcccaatgc

SEQ ID NO: 32 - DNA, 20 bp, Artificial
  RT-PCR primer
ctgagaaaggcggaagtagc

SEQ ID NO: 33 - DNA, 21 bp, Artificial
  RT-PCR primer
ccatagacctgtggctagacg

SEQ ID NO: 34 - DNA, 20 bp, Artificial
  RT-PCR primer
gtgaaggactgtgaccagca

SEQ ID NO: 35 - DNA, 20 bp, Artificial
  RT-PCR primer
gcctgaagtcagccagagtc

SEQ ID NO: 36 - DNA, 20 bp, Artificial
  RT-PCR primer
cgcgtcatccttctttcttc

SEQ ID NO: 37 - DNA, 20 bp, Artificial
  RT-PCR primer
actgggattacaggcgtgag

SEQ ID NO: 38 - DNA, 20 bp, Artificial
  RT-PCR primer
tgcaaatgtcaacgtagcaa

SEQ ID NO: 39 - DNA, 20 bp, Artificial
  RT-PCR primer
caggtccggtagtcatggat

SEQ ID NO: 40 - DNA, 20 bp, Artificial
  RT-PCR primer
tgtttgatgcggaattttca

SEQ ID NO: 41 - DNA, 20 bp, Artificial
  RT-PCR primer
aggaggaaattgaggccaag

SEQ ID NO: 42 - DNA, 20 bp, Artificial
  RT-PCR primer
ggtcacctacccctgttcct

SEQ ID NO: 43 - DNA, 20 bp, Artificial
  RT-PCR primer
ttggggaccttgctgtagtc

SEQ ID NO: 44 - DNA, 20 bp, Artificial
  ChIP primer
ttctccatgttggtcaggtt

SEQ ID NO: 45 - DNA, 20 bp, Artificial
  ChIP primer
agctgcagaacgtattggtc

SEQ ID NO: 46 - DNA, 20 bp, Artificial
  ChIP primer
gcctaagaaccaccctaagc

SEQ ID NO: 47 - DNA, 20 bp, Artificial
  ChIP primer
ttgatacacaaaggcccact

SEQ ID NO: 48 - DNA, 22 bp, Artificial
  ChIP primer
ccgccagtccagtgccgtttct

SEQ ID NO: 49 - DNA, 20 bp, Artificial
  ChIP primer
cgccgcctctcgctgttcct

SEQ ID NO: 50 - DNA, 20 bp, Artificial
  ChIP primer
gtgtctgaaggatggtttgg

SEQ ID NO: 51 - DNA, 20 bp, Artificial
  ChIP primer
tcgcgtgtgtgagagattta

SEQ ID NO: 52 - DNA, 20 bp, Artificial
  ChIP primer
ctcagcctcccaagtagctg

SEQ ID NO: 53 - DNA, 20 bp, Artificial
  ChIP primer
ggaaatcattgtgggattgc

SEQ ID NO: 54 - DNA, 20 bp, Artificial
  ChIP primer
aggaagcaggctgtggtcta

SEQ ID NO: 55 - DNA, 20 bp, Artificial
  ChIP primer
tgtttgatgcggaattttca

SEQ ID NO: 56 - DNA, 20 bp, Artificial
  ChIP primer
gtcacattcaagggccaagt

SEQ ID NO: 57 - DNA, 20 bp, Artificial
  ChIP primer
tttgcctttttggctacgtt

SEQ ID NO: 58 - DNA, 20 bp, Artificial
  ChIP primer
catcacagggtggttgtgag

SEQ ID NO: 59 - DNA, 20 bp, Artificial
  ChIP primer
ccagaaagccccctactttc

SEQ ID NO: 60 - DNA, 20 bp, Artificial
  ChIP primer
gaggatagagaccccaggag

SEQ ID NO: 61 - DNA, 20 bp, Artificial
  ChIP primer
acccctgatctactggaacc

SEQ ID NO: 62 - DNA, 20 bp, Artificial
  ChIP primer
ccactgtcagcacctcttct

SEQ ID NO: 63 - DNA, 20 bp, Artificial
  ChIP primer
agctggtcttgtcaatgagg

SEQ ID NO: 64 - DNA, 20 bp, Artificial
  ChIP primer
ggtggatcacttgaggtcag

SEQ ID NO: 65 - DNA, 20 bp, Artificial
  ChIP primer
gctccttaaacctccctgag

SEQ ID NO: 66 - DNA, 20 bp, Artificial
  ChIP primer
tggagagagaccgaaacaag

SEQ ID NO: 67 - DNA, 20 bp, Artificial
  ChIP primer
cagggtctcactctgtcacc

SEQ ID NO: 68 - DNA, 20 bp, Artificial
  ChIP primer
cacgggaaaaagagagaaca

SEQ ID NO: 69 - DNA, 20 bp, Artificial
  ChIP primer
ctgcacatagtcccagctct

SEQ ID NO: 70 - DNA, 20 bp, Artificial
  ChIP primer
acccacccaatctctagtcc

SEQ ID NO: 71 - DNA, 20 bp, Artificial
  ChIP primer
gacccgttacctctcaggat

SEQ ID NO: 72 - DNA, 20 bp, Artificial
  ChIP primer
atttccatagtgggcatttg

SEQ ID NO: 73 - DNA, 20 bp, Artificial
  ChIP primer
acagtcatctgcggagaaag

SEQ ID NO: 74 - DNA, 20 bp, Artificial
  ChIP primer
gcttccgaccttactccttc

SEQ ID NO: 75 - DNA, 20 bp, Artificial
  ChIP primer
caggaatatcgagagcgtca

SEQ ID NO: 76 - DNA, 20 bp, Artificial
  ChIP primer
ggaaggaaaccgtctagctc

SEQ ID NO: 77 - DNA, 20 bp, Artificial
  ChIP primer
tgtcttggagagcgctagtt

SEQ ID NO: 78 - DNA, 20 bp, Artificial
  ChIP primer
tagacgtgaggggcagatag

SEQ ID NO: 79 - DNA, 20 bp, Artificial
  ChIP primer
agctcacgaggcaaatacac

SEQ ID NO: 80 - DNA, 20 bp, Artificial
  ChIP primer
ccaggagcttaatgaacagg

SEQ ID NO: 81 - DNA, 20 bp, Artificial
  ChIP primer
gggagaacttgacggtcttt

SEQ ID NO: 82 - DNA, 20 bp, Artificial
  ChIP primer
cccttcttcacaggacacac

SEQ ID NO: 83 - DNA, 20 bp, Artificial
  ChIP primer
cgaacctttggtgagagaag

SEQ ID NO: 84 - DNA, 20 bp, Artificial
  ChIP primer
ttcagacactcagggctttc

SEQ ID NO: 85 - DNA, 20 bp, Artificial
  ChIP primer
aaggctggtcttgaactcct

SEQ ID NO: 86 - DNA, 20 bp, Artificial
  ChIP primer
cacgcttataatcccagcac

SEQ ID NO: 87 - DNA, 20 bp, Artificial
  ChIP primer
ctcccacttcagtctctcca

SEQ ID NO: 88 - DNA, 20 bp, Artificial
  ChIP primer
cagcctgggctacagagtaa

SEQ ID NO: 89 - DNA, 20 bp, Artificial
  ChIP primer
cagagtttcgctctttttgc

SEQ ID NO: 90 - DNA, 20 bp, Artificial
  ChIP primer
agctctgggtcagaactgtg

SEQ ID NO: 91 - DNA, 20 bp, Artificial
  ChIP primer
cttggagacgtccctatcaa

SEQ ID NO: 92 - DNA, 20 bp, Artificial
  ChIP primer
tagcaagtgaaagggacagg

SEQ ID NO: 93 - DNA, 20 bp, Artificial
  ChIP primer
actctgctggtctgctgtct

SEQ ID NO: 94 - DNA, 20 bp, Artificial
  ChIP primer
ggaggggcatttttatagga

SEQ ID NO: 95 - DNA, 20 bp, Artificial
  ChIP primer
ggagcctcttacggaaaatc

SEQ ID NO: 96 - DNA, 20 bp, Artificial
  ChIP primer
caccaaacaggcttcttgtc

SEQ ID NO: 97 - DNA, 19 bp, Artificial
  ChIP primer
ggctgagtaaaggggtgtg

SEQ ID NO: 98 - DNA, 20 bp, Artificial
  ChIP primer
aacccttcttcccctgtaac

SEQ ID NO: 99 - DNA, 20 bp, Artificial
  ChIP primer
ccctccttcctgagttgttt

SEQ ID NO: 100 - DNA, 20 bp, Artificial
  ChIP primer
ctttggaaaggggtcttgtt

SEQ ID NO: 101 - DNA, 20 bp, Artificial
  ChIP primer
gatatcagcgggaaatgatg

SEQ ID NO: 102 - DNA, 20 bp, Artificial
  ChIP primer
gaccccaagatctgtgactg

SEQ ID NO: 103 - DNA, 20 bp, Artificial
  ChIP primer
gccaatcagctaaggacaga

SEQ ID NO: 104 - DNA, 20 bp, Artificial
  ChIP primer
tatcagacaccccagacgtt

SEQ ID NO: 105 - DNA, 20 bp, Artificial
  ChIP primer
agagggtcacttttcccttg

SEQ ID NO: 106 - DNA, 20 bp, Artificial
  ChIP primer
ggtctgtctcactgggtttg

SEQ ID NO: 107 - DNA, 20 bp, Artificial
  ChIP primer
ctggcacttggactctccta

SEQ ID NO: 108 - DNA, 20 bp, Artificial
  ChIP primer
gctgggactaggatgaacaa

SEQ ID NO: 109 - DNA, 20 bp, Artificial
  ChIP primer
gaggatccaggtgtgatagc

SEQ ID NO: 110 - DNA, 20 bp, Artificial
  ChIP primer
cctctcatcgtcctcttcct

SEQ ID NO: 111 - DNA, 20 bp, Artificial
  ChIP primer
cagacctgatcggtttctgt

SEQ ID NO: 112 - DNA, 20 bp, Artificial
  ChIP primer
gctgctgaaaagtagggtga

SEQ ID NO: 113 - DNA, 20 bp, Artificial
  ChIP primer
ctagagcttcgggtgtgtgt

SEQ ID NO: 114 - DNA, 20 bp, Artificial
  ChIP primer
gcaggagccaggtatttaca

SEQ ID NO: 115 - DNA, 20 bp, Artificial
  ChIP primer
aagcctccaaagggactaga

SEQ ID NO: 116 - DNA, 20 bp, Artificial
  ChIP primer
gggaggtgagaggttggtat

SEQ ID NO: 117 - DNA, 20 bp, Artificial
  ChIP primer
cagggaagcaggagatgtaa

SEQ ID NO: 118 - DNA, 20 bp, Artificial
  ChIP primer
aggagagtgtggcagatgag

SEQ ID NO: 119 - DNA, 20 bp, Artificial
  ChIP primer
aagtggggtggtttttaagc

SEQ ID NO: 120 - DNA, 20 bp, Artificial
  ChIP primer
aactcgaggtcctggctact

SEQ ID NO: 121 - DNA, 20 bp, Artificial
  ChIP primer
gcagaggaagagacaaccaa

SEQ ID NO: 122 - DNA, 20 bp, Artificial
  ChIP primer
ccatctaaatagccccctgt

SEQ ID NO: 123 - DNA, 20 bp, Artificial
  ChIP primer
cgttttgcttactgccaagt

SEQ ID NO: 124 - DNA, 20 bp, Artificial
  ChIP primer
caagtatctgggaccacagg

SEQ ID NO: 125 - DNA, 20 bp, Artificial
  ChIP primer
ccattcctcaagtcatctgg

SEQ ID NO: 126 - DNA, 20 bp, Artificial
  ChIP primer
ccaagcactcacagaaaacc

SEQ ID NO: 127 - DNA, 20 bp, Artificial
  ChIP primer
ggtagagcgttcagaggaca

SEQ ID NO: 128 - DNA, 20 bp, Artificial
  ChIP primer
ccctttccccagaaataaag

SEQ ID NO: 129 - DNA, 20 bp, Artificial
  ChIP primer
gagatgagggatggaggatt

SEQ ID NO: 130 - DNA, 20 bp, Artificial
  ChIP primer
gcaagtctagcctttgacca

SEQ ID NO: 131 - DNA, 20 bp, Artificial
  ChIP primer
agctatggacccacaatcaa

SEQ ID NO: 132 - DNA, 20 bp, Artificial
  ChIP primer
ggaataaggcatggctcaga

SEQ ID NO: 133 - DNA, 20 bp, Artificial
  ChIP primer
ctgacagcctcaccatctca

SEQ ID NO: 134 - DNA, 22 bp, Artificial
  ChIP primer
tcacattttacaggagcagacg

SEQ ID NO: 135 - DNA, 20 bp, Artificial
  ChIP primer
aaacaaaaggccaacaatgc

SEQ ID NO: 136 - DNA, 20 bp, Artificial
  ChIP primer
gggaaccagatctctcacca

SEQ ID NO: 137 - DNA, 20 bp, Artificial
  ChIP primer
tgtatgtgggtgggtgtgtc

SEQ ID NO: 138 - DNA, 20 bp, Artificial
  ChIP primer
ccaggatccagagagtttgc

SEQ ID NO: 139 - DNA, 20 bp, Artificial
  ChIP primer
gctgagaaggcgtggtagag

SEQ ID NO: 140 - DNA, 20 bp, Artificial
  RT-PCR primer
ttgccccaagtcctcattta

SEQ ID NO: 141 - DNA, 20 bp, Artificial
  RT-PCR primer
aagctgccccatacactgac

SEQ ID NO: 142 - DNA, 20 bp, Artificial
  RT-PCR primer
ttgccccaagtcctcattta

SEQ ID NO: 143 - DNA, 20 bp, Artificial
  RT-PCR primer
aagctgccccatacactgac

SEQ ID NO: 144 - DNA, 20 bp, Artificial
  RT-PCR primer
cctgcaaaaacacagactcg

SEQ ID NO: 145 - DNA, 20 bp, Artificial
  RT-PCR primer
cggtgatttagcagcaagaa

SEQ ID NO: 146 - DNA, 20 bp, Artificial
  RT-PCR primer
ccaggatccagagagtttgc

SEQ ID NO: 147 - DNA, 20 bp, Artificial
  RT-PCR primer
tttaccaaagcatccccaac

SEQ ID NO: 148 - DNA, 20 bp, Artificial
  RT-PCR primer
gggcactggtatcctgaaaa

SEQ ID NO: 149 - DNA, 20 bp, Artificial
  RT-PCR primer
tggagagctggacactgatg

SEQ ID NO: 150 - DNA, 20 bp, Artificial
  RT-PCR primer
cccagaaaaggctcaacatc

SEQ ID NO: 151 - DNA, 20 bp, Artificial
  RT-PCR primer
ttgtcaccatctgcagggta

SEQ ID NO: 152 - DNA, 20 bp, Artificial
  RT-PCR primer
gcagcagaagaagcctttgt

SEQ ID NO: 153 - DNA, 20 bp, Artificial
  RT-PCR primer
aatgccagggagaacacagt

SEQ ID NO: 154 - DNA, 20 bp, Artificial
  RT-PCR primer
cagtggcctagtgagcatga

SEQ ID NO: 155 - DNA, 20 bp, Artificial
  RT-PCR primer
aaacaggtagtcggggatca

(19) United States
(12) Patent Application Publication
(10) Pub. No.: US 2007/0292857 A1
(43) Pub. Date: Dec. 20, 2007

(54) Mapping histone modifications by DNA microarray

(76) Inventors: Rama Natarajan, Hacienda Heights, CA (US); Feng Miao, Arcadia, CA (US)

Correspondence Address: PERKINS COIE LLP, POST OFFICE BOX 1208, SEATTLE, WA, 98111-1208, US

(21) Appl. No.: 11/501,662

(22) Filed: Aug. 8, 2006

Related U.S. Application Data
(60) Provisional application No. 60/706,410, filed on Aug. 8, 2005.

(51) Int. Cl. C12Q 1/68

(52) U.S. Cl. 435/6

(57) ABSTRACT

Many human diseases are now realized to have epigenetic features. Post-translational modification of histones plays a major role in these epigenetic features. Chromatin immunoprecipitation assay (ChIP) is currently the method of choice for localizing histone modifications in a step-wise fashion. This technique utilizes histone modification-specific antibodies to enrich DNA (ChIP DNA), followed by the use of promoter-specific primers to localize the modification. Methods of using DNA microarrays to screen ChIPed DNA are provided herein.

### CROSS-REFERENCE TO RELATED APPLICATION

The present application claims priority to U.S. Provisional Application No. 60/706,410, filed Aug. 8, 2005, the disclosure of which is incorporated by reference herein in its entirety.

### GOVERNMENT INTEREST

This invention was made with government support in part by grants from the National Institutes of Health, specifically NIDDK RO1 DK065073. The government may have certain rights in this invention.

### REFERENCE TO TABLES SUBMITTED ON COMPACT DISC

Tables 1, 2, and 4-10 are contained on a CD-ROM provided herewith. Two copies of this CD-ROM are provided. Any reference to these tables in the specification should be considered an incorporation by reference of the contents of the table at that particular place in the specification.

### BACKGROUND

Eukaryotic chromosomes are composed of chromatin, a nucleoprotein complex consisting of genomic DNA, proteins, and a small amount of RNA. Chromatin is organized into a series of tightly coiled protein-DNA structures called nucleosomes, with each nucleosome consisting of approximately 150 nucleotides of DNA wrapped around an octamer of histone proteins. This octamer consists of two copies each of the histone proteins H2A, H2B, H3, and H4 (Luger 1997). Denser regions of chromatin are classified as heterochromatin, while less dense regions are classified as euchromatin. Gene transcription and activation are dynamic processes in which inaccessible, repressive heterochromatin is converted into accessible euchromatin, allowing access to transcription factors and the basal transcription machinery.

It is now clear that apart from the binding of transcription factors to their cognate promoter sites, transcriptional activation or repression is tied to the recruitment of protein complexes that alter chromatin structure via enzymatic modification of histones and/or nucleosome remodeling. The C-terminal domains of histones play a key role in nucleosome assembly by mediating histone-histone and histone-DNA interactions, while the N-terminal domains make contact with DNA and adjacent nucleosomes. The N-terminal domains of histone proteins H3 and H4 are subject to a variety of modifications, including methylation, acetylation, phosphorylation, ubiquitination, and ADP-ribosylation (Zhang 2001; Sims 2003). These modifications occur especially at lysine, serine, and arginine residues. The subtle interrelationship and potential functional significance of these modifications are only just beginning to be understood. Research into these modifications has led to the realization that chromatin is not merely a scaffolding structure, but also a dynamic entity capable of regulating gene expression and cellular functions. This has led to the notion of the “histone code,” which postulates that the type and number of histone N-terminal tail modifications serve as an epigenetic regulatory mechanism governing specific transcriptional states and biological outcomes (Strahl 2000; Turner 2000; Jenuwein 2001; Turner 2002; lizuka 2003). Studies supporting the notion of a “histone code” have demonstrated that histone modifications participate in a variety of essential biological processes, including gene activation, gene silencing, gene repression, and X chromosome inactivation (Lachner 2002; Lachner 2003). The histone code may be characterized by single histone modifications or by patterns of histone modifications (Berger 2002). Despite the advances that have been made in understanding histone modifications in recent years, the distribution pattern of histone modifications in human genes and their role in transcriptional regulation is still largely unknown.

### SUMMARY

In certain embodiments, methods are provided for non-invasive mapping of histone modifications in a subject. In certain of these embodiments, a blood sample from a subject is subjected to a chromatin immunoprecipitation (ChIP) assay using one or more antibodies that bind to one or more specific histone modifications, and the genomic location of these specific histone modifications is identified using a DNA microarray. In some embodiments, the DNA microarray may be a cDNA microarray. In certain embodiments, one or more of the histone modifications may be methylations. In certain embodiments, the histone modifications may be located in gene coding regions.

In certain embodiments, further methods are provided for non-invasive mapping of histone modifications in a subject. In certain of these embodiments, a blood sample from a subject is divided into a chromatin immunoprecipitation sample and a control sample, and the chromatin immunoprecipitation sample is subjected to a ChIP assay using one or more antibodies that bind to one or more specific histone modifications. DNA isolated by this method is radiolabeled with a first label, DNA from the control sample is radiolabeled with a second label, and the chromatin immunoprecipitation and control samples are combined and applied to a human cDNA microarray. The ratio of the intensity of the first radiolabel to the intensity of the second radiolabel is calculated for each probe on the microarray, and a set of one or more genes with a ratio greater than or equal to a certain cut-off value are identified as candidate genes associated with one or more histone modifications. In certain of these embodiments, the first or second label may be Cy5-dCTP or Cy3-dCTP. In some embodiments, the cut-off value is 2.0. In certain embodiments, one or more of the histone modifications may be methylations. In embodiments wherein one or more of the histone modifications are methylations, methylation may occur at, for example, H3-K4, H3-K9, H4-K20, H3-K27, H3-K36, and/or H3-K79.

In certain embodiments, methods are provided for diagnosing type 1 diabetes in a subject. In certain of these embodiments, a test blood sample from a subject and a control blood sample from a known healthy subject are both subjected to a ChIP assay using an antibody that binds H3-K9Me2. DNA isolated from the test sample by this method is radiolabeled with a first label, DNA isolated from the control sample by this method is radiolabeled with a second label, and the test sample and control sample are combined and applied to a microarray containing probes from one or more of the genes listed in Tables 11 and 12. The ratio of the intensity of the first radiolabel to the intensity of the second radiolabel is calculated for each probe on the microarray, with a label ratio of 2.0 or greater or 0.5 or lesser indicating a diagnosis of type 1 diabetes.

In certain embodiments, methods are provided for determining whether a subject is at risk for developing type 1 diabetes. In certain of these embodiments, a test blood sample from a subject and a control blood sample from a known healthy subject are both subjected to a ChIP assay using an antibody that binds H3-K9Me2. DNA isolated from the test sample by this method is radiolabeled with a first label, DNA isolated from the control sample by this method is radiolabeled with a second label, and the test sample and control sample are combined and applied to a microarray containing probes from one or more of the genes listed in Tables 11 and 12. The ratio of the intensity of the first radiolabel to the intensity of the second radiolabel is calculated for each probe on the microarray, with a label ratio of 2.0 or greater or 0.5 or lesser indicating that the subject is at risk for developing type 1 diabetes.

In certain embodiments, methods are provided for determining whether a subject with type 1 diabetes is at risk for accelerated development of vascular complications. In certain of these embodiments, a test blood sample from a subject and a control blood sample from a known healthy subject are both subjected to a ChIP assay using an antibody that binds H3-K9Me2. DNA isolated from the test sample by this method is radiolabeled with a first label, DNA isolated from the control sample by this method is radiolabeled with a second label, and the test sample and control sample are combined and applied to a microarray containing probes from one or more of the genes listed in Tables 11 and 12. The ratio of the intensity of the first radiolabel to the intensity of the second radiolabel is calculated for each probe on the microarray, with a label ratio of 2.0 or greater or 0.5 or lesser indicating that the subject is at risk for accelerated development of vascular complications.

### BRIEF DESCRIPTION OF THE DRAWINGS

FIG. 1: ChIP/cDNA microarray analysis of H3-K4 and H3-K9 methylation in human coding regions. A. Virtual microarray images of H3-K4Me2 and H3-K9Me2 patterns. B. Venn diagram comparing candidate genes from H3-K4Me2/H3-K9Me2 and H3-K4Me2/H3-K4Me3 data sets. Venn diagram analyses were carried out using GeneSpring. C. Conventional ChIP analysis using select candidate genes from H3-K4Me2 and H3-K9Me2 data sets. D. RT-PCR analysis of expression levels of select genes from H3-K4Me2 and H3-K9Me2 data sets.

FIG. 2: Venn diagram comparing candidate genes from H3-K4 dimethylation and H3-K9 dimethylation data sets from human promoter regions.

FIG. 3: ChIP/cDNA microarray analysis of H3-K79 and H3-K36 methylation in human coding regions. A. Venn diagram comparing candidate genes from H3-K4Me2, H3-K36Me2, and H3-K79Me2 data sets. B. Conventional ChIP analysis using select candidate genes from H3-K4Me2, H3-K36Me2, and H3-K79Me2 data sets. C. RT-PCR analysis of expression levels of select genes from the H3-K36Me2 and/or H3-K79Me2 data sets. D. Conventional ChIP analysis of H3-K4 and H3-K36 dimethylation in multiple sections of the transcribed regions of CD37, ICAM3, KLF1, and MCAM. The upper panel shows the regions of each gene that were amplified. Numbers indicate locations of the PCR primers.

FIG. 4: Venn diagram comparing candidate genes from H3-K9/K14 acetylation and various H3 methylation data sets from human coding regions. B. Conventional ChIP analysis of H3-K9/14 acetylation in select methylated genes.

FIG. 5: ChIP/cDNA microarray analysis of H3-K27, H4-K20, and H3-K9 methylation in human coding regions. A. Venn diagram comparing candidate genes from H3-K9Me3, H3-K27Me2, and H4-K20Me2 data sets. B. Conventional ChIP analysis using select candidate genes H3-K9Me3, H3-K27Me2, and H4-K20Me2 data sets. C. RT-PCR analysis of expression levels of select genes H3-K9Me3, H3-K27Me2, and H4-K20Me2 data sets.

FIG. 6: Conventional ChIP analysis of H3-K9 methylation in high glucose versus no glucose cells and Type 1 diabetes patients versus normal subjects. ChIP was performed using four candidate genes from the 6p21.3 locus.

FIG. 7: Genes associated with altered H3 methylation patterns in Type 1 diabetes patients. Left panel: genes that exhibit decreased H3 methylation in Type 1 diabetes patients versus normal subjects. Right panel: genes that exhibit increased H3 methylation in Type 1 diabetes patients versus normal subjects.

### DETAILED DESCRIPTION

The following detailed description is merely intended to illustrate various embodiments of the present invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Definitions

The phrase “histone modification” as used herein refers to a covalent modification of the N-terminal tail of a histone protein. Examples of histone modifications include, for example, methylation, acetylation, phosphorylation, ubiquitination, and ADP-ribosylation.

The phrase “mapping histone modifications” as used herein refers to the identification of genes that are associated with a particular histone modification. Genes may be identified that are associated with a particular modification on a specific residue in a single histone protein. For example, genes may be identified that are associated with a specific methylation state (e.g., di-, trimethylation, etc.) at a specific lysine residue on a histone protein.

The phrase “blood sample” as used herein refers to a sample obtained from a subject that contains one or more types of blood cells. A blood sample may be either whole blood or a mixture of one or more purified blood cells. For example, a blood sample may consist of purified white blood cells, such as for example purified monocytes or lymphocytes.

### Abbreviations

The following abbreviations are used herein: Ac, acetylation; ChIP, chromatin immunoprecipitation assay; GWLA, genome-wide location analysis; HMT, histone methyl transferase; K, lysine; LPS, lipopolysaccharide; Me, methylation; Me2, dimethylation; Me3, trimethylation; ORF, open reading frame; TNF, tumor necrosis factor.

### Mapping Histone Modifications

Heterochromatin and euchromatin have been known for some time, but a flurry of recent findings has changed the view of chromatin and its role in cellular functions (van Leeuwen 2002). Histone modifications are integral parts of the genetic information, and elucidating the biological and functional relevance of these modifications is imperative to a complete understanding of chromatin status and function. The present disclosure provides novel data suggesting that chromatin regions can be classified based on their histone modification patterns. These patterns not only define chromatin features such as heterochromatin and euchromatin, but also define whether particular genes are expressed, repressed, or silenced. Decoding of histone methylation patterns will allow the identification of information about epigenetic changes that complements the genetic information obtained from the human genome sequence. In the present disclosure, all types of histone methylations were identified in human coding regions, but not all possible combinations of histone methylations were observed. This indicates that histone methylation does not distribute randomly, and that specific histone methylation patterns may have important biological significance.

Histone acetylation and deacetylation, mediated by coactivators and repressors respectively, has been known for some time to play a major role in gene expression (Roth 2001). Acetylated histones confer accessibility of the DNA template to the transcriptional machinery, while deacetylation of histones by histone deacetylases (HDACs) serves to repress transcription. Histone methylation, on the other hand, has only recently been identified as cooperating with other factors to alter chromatin structure and regulate transcription either positively or negatively (Wu 2000). Unlike histone acetylation or phosphorylation, histone methylation has generally been considered stable or “irreversible,” with no histone demethylase enzymes being identified yet. However, recent data indicate that histone methylation may also have dynamic features, in which the methyl group is removed by an active process necessary for regulated gene expression (Ma 2001; Bannister 2002; Saccani 2002). Histone methylation may be linked to transcriptional activation, repression, or silencing of a specific gene, depending on the precise methylation site and state of the histone proteins associated with that gene (Lachner 2001; Litt 2001; Noma 2001). Thus, changes in histone methylation status may function to switch a gene from one state to another.

Recent studies have provided convincing evidence that histone modifications in gene coding regions, in addition to those in gene promoter regions, can play an important role in gene regulation and expression. For example, it has been shown in yeast that there is more histone H3-K4 methylation in coding regions than in promoter regions (Bernstein 2002). Some experiments have suggested that histone methylation in gene coding regions may be a marker for active genes (Bernstein 2002; Santos-Rosa 2002).

Transcriptionally active euchromatin can be methylated at H3-K4, H3-K36, and H3-K79, while transcriptionally repressed euchromatin can be methylated at H3-K9, H3-K27, and H4-K20 (Sims 2003). More importantly, several specific histone methylation patterns have been identified in heterochromatin (Plath 2003; Silva 2003; Okamoto 2004; Schotta 2004). Mammalian histone methyl transferases (HMTs) have been identified that specifically methylate histones at H3-K4 (Wang 2001; Goo 2003), H3-K9 (Rea 2000; Kuzmichev 2002; Muller 2002; Orlando 2003), H3-K36 (Rayasam 2003), H3-K79 (Feng 2002), and H4-K20 (Nishioka 2002). Histone methylation may occur in a mono-, di-, or tri-methylated state (Czermin 2002; Kuzmichev 2002; Santos-Rosa 2002; Tamaru 2003), and the extent of methylation appears to correlate with the degree of gene activation or silencing (Sims 2003).

It has been proposed that methylation of H3-K4 and H3-K79 marks active chromatin and limits silencing to discrete domains by preventing the promiscuous binding of Sir proteins that help maintain an epigenetic silenced state and the formation of heterochromatin in yeast (van Leeuwen 2002a; van Leeuwen 2002b). Previous studies have also revealed that methylation of H3-K79 is required for recruitment of Sir protein (Ng 2003), and that H3-K79 methylation is regulated during the cell cycle (Feng 2002). However, it is still unclear what determines active coding regions to be regulated by H3-K4 or H3-K36 methylation, why these regions are marked by specific dual methylation patterns, and what the biological functions of these methylation patterns are.

Dynamic changes in H3-K9 methylation have been observed to occur in response to lipopolysaccharide (LPS) at a subset of inflammatory genes in dendritic cell, and these changes are inversely correlated with RNA polymerase II recruitment (Saccani 2002). Thus, dynamic modulation of H3-K9 methylation adds an additional regulatory level to the transcription of tightly controlled inducible inflammatory genes (Saccani 2002; Bannister 2002). Since it is known that diabetic conditions can induce inflammatory genes in monocytes (Morohoshi 1995; Hofmann 1998; Guha 2000; Jain 2002; Cipollone 2003; Shanmugam 2003a; Shanmugam 2003b), it is possible that histone methylation provides a key gene regulatory mechanism in diabetes.

Type 1 diabetes (T1D) is an autoimmune disorder in which the body attacks and destroys the insulin-producing beta cells of the pancreas. Microvascular complications associated with T1D include retinopathy, neuropathy, and nephropathy. The Diabetes Control and Complications Trial (DCCT) demonstrated the benefit of strict glycemic control with intensive treatment relative to conventional treatment to reduce the progression of multiple microvascular complications of diabetes. The subsequent Epidemiology of Diabetic Interventions and Complications (EDIC) study provided a follow-up study of DCCT patients given intensive treatment (IT) versus those given conventional treatment (CT). During the EDIC study, all patients were subjected to IT, and both gradually achieved similar glycemic controls. However, it was noted that patients given IT during the DCCT retained the benefits of that IT, and thus exhibited significantly lower rates of diabetic complications than the other group. This phenomenon is referred to as “hyperglycemic memory.”

Biochemical mechanisms and key factors that have been implicated in T1D-induced complications include hyperglycemia (Pugliese 1991; Ruderman 1992), oxidant stress (Baynes 1991; Ido 1997), advanced glycation end products (AGEs) (Brownlee 1988; Schmidt 1994), protein kinase C (PKC) (Ishii 1998), and mitochondrial superoxide production (Nishikawa 2000). Numerous studies have been performed to identify T1D susceptibility genes. More than 20 candidate genes or chromosomal regions have been identified, and the list of new loci associated with T1D continues to grow (Florez 2003). Many of these T1D susceptibility genes have not been fully confirmed yet, mainly due to size effects. However, there are well studied and convincing associations to loci such as HLA region (6p21.3), insulin VNTR, and CTLA4. Despite this work, there are still numerous gaps in knowledge with regards to the genome-wide aberrant regulation of key genes under diabetic conditions. The failure to identify and confirm additional candidate genes suggests that epigenetic changes such as nucleosomal modifications may play an important role in the disease process. In particular, histone methylation in coding or promoter regions may lead to repression of certain “protective” genes or activation of “harmful” genes, leading to the initiation of T1D or worsening of its complications. Understanding of the role of these modifications may help explain the “hyperglycemic memory” phenomenon.

Genome-wide location analysis (GWLA) is a tool that combines chromatin immunoprecipitation (ChIP) with the use of genomic DNA microarrays to monitor protein-DNA interactions across the entire genome (Ren 2000). Substantial information has been obtained about gene transcription networks by performing GWLA using transcription factors (Li, Z. 2003; Martone 2003; Odom 2004). Recently, GWLA has been utilized to map histone acetylation and/or methylation in yeast and Drosophila (Bernstein 2002; Ng 2002; Robyr 2002; Schubeler 2004). The application of this technique to map human histone modifications, however, has been hampered by the fact that the majority of available human DNA microarrays are cDNA microarrays. Unlike in yeast and Drosophila, human whole genome microarrays are not yet well developed.

Human cDNA microarrays have been used for comparative genomic hybridization (CGH) analysis, where they have provided very high mapping resolution for radiometric measurement of variation in genomic DNA copy number (Pollack 1999). cDNA microarrays provide an attractive plafform for CGH analysis because they are well characterized and widely used. The present disclosure utilizes cDNA microarrays to map histone methylation and acetylation in human gene coding regions. The examples described herein demonstrate the feasibility of mapping human histone modifications using a ChIP/cDNA microarray approach.

ChIP/cDNA microarray analysis was initially used to map H3-K4 and H3-K9 dimethylation in human THP-1 monocytes. THP-1 cells were selected because they are of monocytic lineage, and studies using this cell type can potentially be extrapolated to all human blood cells. ChIP was performed using antibodies against H3-K4Me2, H3-K4Me3, and H3-K9Me2, and ChIPed DNA samples were amplified by ligation-mediated PCR. ChIPed samples were labeled with Cy5-dCTP, while control DNA samples were labeled with Cy3-dCTP. ChIPed samples and control samples were mixed and hybridized to a human CDNA microarray. Following signal normalization, candidate genes were selected that exhibited a 635/532 nm ratio of 2.0 or greater. This ratio corresponds to the ratio of Cy5 to Cy3 signal intensity. Results from these experiments indicated that H3-K4Me2 and H3-K9Me2 are generally mutually exclusive, and that H3-K4Me2 and H3-K4Me3 are closely correlated to one another. Out of 223 H3-K4Me2 candidate genes and 213 H3-K9Me2 candidate genes, only ten genes appeared in both groups. When the experiments were repeated using a promoter microarray rather than a cDNA microarray, similar results were obtained. This is consistent with previous single-gene experiments showing that promoters methylated at H3-K9 are generally not methylated at H3-K4, and vice versa. Conventional ChIP using selected candidate genes verified these results, confirming that a ChIP/cDNA microarray approach is a reliable method for mapping histone modifications. RT-PCR experiments were then conducted to determine the correlation between methylation of H3-K4 and H3-K9 and the expression of candidate genes. As expected, candidate genes associated with H3-K4Me2 were clearly expressed, while candidate genes associated with H3-K9Me2 were either not expressed or expressed at very low levels, suggesting that H3-K4Me2 and H3-K9Me2 have opposite functions.

ChIP/cDNA microarray experiments were repeated using THP-1 cells and antibodies against H3-K4Me2, H3-K79Me2, and H3-K36Me2. Each of these methylation marks had previously been associated with active gene coding region. The experiments showed that H3-K4Me2 and H3-K79Me2 are closely associated, which supports the notion that H3-K4Me2/H3-K79Me2 represents a specific methylation pattern associated with active euchromatin. H3-K79Me2 was also found to be closely associated with H3-K36Me2, suggesting a novel H3-K36Me2/H3-K79Me2 methylation pattern. This result was confirmed by performing conventional ChIP on selected H3-K36Me2 and H3-K79Me2 candidate genes. RT-PCR confirmed that each of these candidate genes was expressed. Despite the correlations observed between H3-K4Me2 and H3-K79Me2 and between H3-K36Me2 and H3-K79Me2, no correlation was observed between H3-K4Me2 and H3-K36Me2. This suggests that H3-K4Me2 and H.3-K36Me2 serve as distinctly different marks for active coding regions.

Previous experiments have suggested that H3-K4 methylation is biased towards the 5′ end of genes, while H3-K36 methylation is biased towards the 3′ end. Thus, the observed differences in H3-K4 and H3-K36 methylation patterns may simply reflect which portion of a particular gene is represented on the microarray. On the other hand, dual methylation patterns such as H3-K4/K79 have also been reported in Drosophila (Schubeler 2004). The present results, coupled with the H3-K9/H4-K20 methylation index model proposed previously (Schotta 2004), suggest that H3-K79 methylation may index active chromatin domains by association with H3-K4 or H3-K36 methylation.

Several lines of evidence have indicated that the rate of transcriptional elongation mediated by RNA polymerase II is regulated in part by methylation at H3-K4, H3-K36, and H3-K79 through at least two mediator complexes, PAF and COMPASS (Gerber 2003; Hampsey 2003; Shilatifard 2004). The establishment of a mature elongation complex involves the successive recruitment of specific factors, which is coordinated by the carboxyl-terminal domain of TFIIH. This domain contains a heptapeptide repeat with the consensus sequence Tyr-Ser-Pro-Thr-Ser-Pro-Ser. Interestingly, it has been reported that the kinase Kin28 phosphorylates serine-5 in this repeat to coordinate the recruitment of the H3-K4 methylase Set1 (Ng 2003). Likewise, the Ctk1 kinase phosphorylates serine-2 to coordinate the recruitment of the H3-K36 methylase Set 2 (Li, B. 2003). These observations support the conclusion that H3-K4 and H3-K36 methylations are distinctly different marks for active coding regions that may be mediated by different kinases.

On the other hand, Set2 was shown to co-purify with RNA polymerase II. Synthetic genetic array analysis reveals that Set2 interacts with many proteins, including five subunits of the PAF complex (Leo1, Ctr9, Rtf1, Ccd73, and Paf1) and elongation factors Soh1 and Chd1 (Krogan 2003a). Methylation of H3-K79 by Dot1p also requires the PAF complex. Set2 and Dot1p both interact with Rtf1 and Paf1 genetically. Furthermore, methylation of both H3-K36 and H3-K79 is regulated by the PAF complex (Krogan 2003a; Krogan 2003b). Together, these data provide strong evidence in support of the present finding that H3-K36 and H3-K79 methylations are closely related and associated with active gene transcription.

Furthermore, H3-K4 and H3-K79 methylation in yeast requires ubiquitination of lysine 123 of histone H2B in a process involving the ubiquitin-conjugating enzyme Rad6 (Dover 2002; Sun 2002), while H3-K36 methylation does not. Although there is currently no biochemical evidence supporting the interaction of Dot1 and RNA polymerase II via the PAF complex, the striking similarities between regulation of Dot1-mediated H3-K79 methylation and COMPASS-mediated H3-K4 methylation suggest a similar mechanism by which Dot1 is recruited to chromatin. The association between H3-K4 and H3-K79 methylation suggests that there could be an interaction between Dot1 and one or more proteins in COMPASS, or that this interaction could be mediated through the PAF complex. Since H3-K79 methylation is associated with H3-K36 methylation, it is also possible that there is an interaction between Dot1 and NSD1, or that this interaction is mediated through the PAF complex.

ChIP/cDNA microarray experiments were repeated using THP-1 cells and antibodies against H3-K9Me3, H4-K20Me2, and H3-K27Me2. These experiments revealed a close correlation between each of these methylation marks, and the results were verified by performing traditional ChIP using select candidate genes. Although H3-K9Me2 is a known gene silencing marker, RT-PCR revealed that these candidate genes were not necessarily silenced. This suggests that the consequence of the H3-K9Me3/H4-K20Me2/H3-K27Me2 methylation pattern is different than that of H3-K9Me3 alone, perhaps as a result of heterozygous methylation (i.e., only one of two gene copies is methylated). Additional experiments established that genes associated with each of these three methylation markers are hypoacetylated throughout the genome.

H3-K9, H4-K20, and H3-K27 methylation occur only in higher eukaryotes, not in yeast (Sims 2003). Unlike methylation at H3-K4, H3-K36, or H3-K79, there is no evidence that methylation at H3-K9, H4-K20, and H3-K27 is associated with RNA polymerase II. Several reports indicate that certain histone methyltransferases, including those acting at H3-K9, H3-K27, and H4-K20, can repress gene transcription by creating a docking site for a repressor protein that recruits a core repressor complex. H3-K9 and H3-K27 methylation are involved in Hox gene silencing, X inactivation, germline development, stem cell pluripotency, and cancer metastasis. The models of each of these silencing mechanisms are different, but each involves the EED-EZH2 complex (Cao 2002). Based on the pattern of H3-K9Me3, H3-K27Me2, and H4-K20Me2 observed herein, it is likely that candidate genes associated with these methylation markers are regulated by the PcG/TrxG complex (Cao 2002; Czermin 2002; Kuzmichev 2002; Muller 2002; Orlando 2003; Silva 2003; Kirmizis 2004). This idea is supported by previous studies showing that H3-K9Me3 and H3-K27Me2 are linked to PcG controlled silencing (Cao 2002; Czermin 2002; Orlando 2003), as well as the present identification of two H3-K9Me3 candidate genes (Trip-Br2 and CNTFR) that are PcG component SUZ12 target genes (Kirmizis 2004). Since PcG and TrxG complexes can compete with one another at the same target gene to determine whether the gene is silenced or expressed, the final outcome will depend in part on the concentration of PcG or TrxG complexes and the methylation patterns of the target gene. If the competition between PcG and TrxG reaches a balance, one copy of the target gene may by occupied by the PcG complex and silenced while the other is occupied by TrxG and actively transcribed, although both copies exhibit the H3-K9Me3/H3-K27Me2/H4-K20Me2 pattern. Another possibility is that H3-K9Me3 and H3-K27Me2 are markers for a silenced locus while H4-K20Me2 is a marker for an active locus, since ASH 1, a component of TrxG, can methylate H4-K20 and H3-K4 (Beisel 2002). A slight overlap was observed in the present disclosure between H3-K4Me2 and H4-K20Me2. However, in this scenario, H3-K4Me2 would be an active mark. The allele-specific gene expression model has been associated with epigenetic phenomena of X chromosome inactivation and genomic imprinting. By considering PcG/TrxG participation, the allele-specific gene expression model may explain the present observation that certain genes exhibiting the H3-K9Me3/H3-K27Me2/H4-K20Me2 pattern are still transcriptionally active.

The fact that H3-K9, H3-K27, and H4-K20 methylation was found to occur in hypoacetylated coding regions suggests that histone deacetylases and related protein complexes are involved in the establishment and maintenance of methylation at these sites. This is consistent with the notion that H3-K9, H3-K27, and H4-K20 methylation are associated with gene silencing (Zhang 2001). H3-K9 methylation has been studied extensively, and the current model of establishing and maintaining H3-K9Me includes the following steps: 1) H3-K9 is deacetylated by specific histone deacetylases; 2) H3-K9 is methylated by a histone methyltransferase such as G9a or Suv39H1; 3) HP1 recognizes methylated H3-K9 and propagates methylation by recruiting histone methyltransferases (Sims 2003). The mechanism underlying methylation of H3-K27 and H4-K20 is not yet clear, but it has been suggested that the H3-K27 mechanism may be similar to that of H3-K9 (Sims 2003).

A number of studies have shown that euchromatin H3-K9Me2 regulated by G9a is essential for early embryogenesis and is involved in the transcriptional repression of developmental genes (Tachibana 2002). G9a is a major force of euchromatin silencing (Rice 2003). The results disclosed herein reveal a substantial number of genes that are repressed by H3-K9Me2. This supports the idea that H3-K9Me2 plays a major role in histone methylation-mediated transcriptional repression at the gene level.

It has been reported previously that H3-K9, H3-K20, and H3-K27 methylation are enriched at pericentric, centromeric, and telomeric regions of chromatin, and also on imprinted genes (Sims 2003; Schotta 2004). Several of the methylated candidate genes identified herein are also located at these regions. For example, SERPING1 is found around centromeric regions, MLLT4 is found around telomeric regions, and the imprinted gene IGF2R was identified as a candidate gene.

ChIP/cDNA microarray experiments were repeated using THP-1 cells and antibodies against H3-K9/K14Ac. These experiments revealed a close correlation between H3-K9/K14Ac and H3-K4Me2, H3-K4Me3, H3-K36Me2, and H3-K9Me2, and a poor correlation between H3-K9/K14Ac and H3-K9Me2, H3-K9Me3, H3-K27Me2, and H4-K20Me2. These results are consistent with the notion that H3-K4, H3-K36, and H3-K79 methylation are associated with active transcription, and H3-K9, H3-K27, and H3-K79 methylation are associated with regions that are not being actively transcribed.

ChIP/cDNA microarray assays were performed using antibodies against H3-K9Me2 to identify changes in histone methylation patterns associated with diabetes. Candidate genes associated with H3-K9Me2 were identified in THP-1 cells cultured in high glucose versus THP-1 cells cultured in normal glucose. A set of genes was identified that were associated with changes in H3-K9Me2 levels under high glucose conditions. Similar experiments were performed using monocytes and lymphocytes from normal subjects versus subjects with type 1 diabetes. Again, a set of genes was identified that displayed changes in H3-K9Me2 in type 1 diabetes. This information was used to identify genomic “hotspots” associated with type 1 diabetes. The experiments were also repeated using promoter microarrays, leading to similar results. Similar experiments may be performed to compare histone methylation patterns in diabetic subjects given IT treatment versus CT treatment. This information can be used to identify methylation patterns associated with therapeutic effectiveness.

These results illustrate the utility of the methods disclosed herein as means for identifying histone methylation patterns associated with diabetes and other metabolic disorders. Information obtained using this method can be used to diagnose diabetic conditions, assess disease progression, assess the effectiveness of therapeutic approaches, or predict the likely outcome or effectiveness of a particular therapeutic approach. In addition, this information can be used in conjunction with various therapeutic approaches to more specifically tailor treatment to a particular subject. It may also be used to develop novel therapeutic approaches, such as targeted histone methylation or dimethylation to alter gene expression patterns associated with the disease.

The examples presented herein illustrate the power of the claimed methods to map histone modifications in human gene coding regions and to rapidly profile thousands of genes to determine the relevance of key histone modification profiles both individually and globally. These examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### ChIP/cDNA Microarray Analysis of H3-K4 and H3-K9 Dimethylation

Histone H3-K4 methylation and H3-K9 methylation are considered hallmarks of euchromatin and heterochromatin, respectively. In addition, it is widely accepted that H3-K4 methylation correlates with active genes, while H3-K9 methylation correlates with inactive or silenced genes (Sims 2003).

ChIP/cDNA microarray analysis was implemented in human THP-1 monocytes using antibodies against dimethylated H3-K4 (H3-K4Me2) and dimethylated H3-K9 (H3-K9Me2) (Upstate, Lake Placid, N.Y.). Cells were maintained as described (Miao 2004) in RPMI 1640 medium containing 10% heat-inactivated fetal calf serum (FCS), 10 mM HEPES, 2 mM glutamine, 50 μg/ml streptomycin, 50 units/ml penicillin, 50 82 M β-mercaptoethanol, and 5.5 mM glucose in a 5% CO₂ incubator at 37° C.

ChiPs were performed using described protocols (Ren 2000; Miao 2004) with some modifications. Briefly, 5×10⁷ THP-1 cells were crosslinked in 1% formaldehyde for 40 minutes to generate protein-protein and protein-DNA crosslinks (Solomon 1985; Weinmann 2002; Egger 2004), washed twice in PBS, and resuspended in lysis buffer (1% SDS, 10 mM EDTA, 50 mM Tris-HCL (pH 8.1) and 1× protease inhibitor cocktail) (Roche Molecular Biochemicals, Penzberg, Germany). Resuspended cells were sonicated one to three times for 30 seconds each at 40% maximum setting (Model 250 Sonicator; Branson Ultrasonics, Danbury, Conn.), then centrifuged for 10 minutes. One-fifth of the lysate was kept as a genomic DNA control. Supernatants were collected and diluted in buffer (1% Triton X-100, 2 mM EDTA, 150 mM NaCl, 20 mM Tris-HCl (pH 8.1), and 1× protease inhibitor cocktail), then immunocleared with 5 μg sheared salmon sperm DNA for 1 hour at 4° C. Immunoprecipitation was performed for 15 hours at 4° C. with 10-20 μg of Protein-A sepharose pre-coupled to H3-K4Me2 or H3-K9Me2 primary antibodies. Precipitates were washed twice with distilled water and extracted twice with 1% SDS containing 0.1 M NaHCO3. Elutes were pooled and heated at 65° C. for at least 6 hours to reverse formaldehyde crosslinking.

ChIPed DNA samples were amplified by ligation-mediated PCR. DNA was blunted with T4 DNA polymerase, purified, and ligated with linkers (SEQ ID NO: 1 and SEQ ID NO: 2) overnight at 16° C. After purification on Qiagen Spin columns (Qiagen, Valencia, Calif.), one-fourth of the DNA was used for PCR amplification (20 cycles) using the primer set forth in SEQ ID NO: 3. PCR products were purified on Qiagen Spin columns and eluted with 100 μl TE buffer. 200 ng of amplified DNA was mixed with 20 μl of 2.5× random primer solution (Invitrogen, Carlsbad, Calif.) and distilled water to a final volume of 42.5 μl. This mixture was boiled for 5 minutes, cooled on ice for 5 minutes, and then incubated with 5 μl of 10× low dCTP nucleotide mixture (2.5 mM dATP, 2.5 mM dTTP, 2.5 mM dGTP, and 0.6 mM dCTP), 40 units of Klenow DNA polymerase (Invitrogen), and 2.5 μl of Cy5-dCTP (Perkin Elmer, Boston, Mass.) for 8 hours at 37° C. Labeled DNA was concentrated to 25 μl using an Amicon Microcon YM-30 (Millipore, Bedford, Mass.). The genomic DNA control was labeled with Cy3-dCTP using a similar procedure.

In a new Eppendorf tube, Cy5-labeled ChIPed DNA was mixed with Cy3-labeled genomic DNA, 30 μg human Cot-1 DNA (Invitrogen), 100 μg yeast total tRNA (Roche), and 20 pg poly-dA-dT (Sigma-Aldrich, St. Louis, Mo.). This mixture was adjusted to contain 3.4×SSC and 0.3% SDS in a final volume of 100 μl, heated at 95° C. for 5 minutes to denature, and cooled at 25° C. for 5 minutes, then gently spotted onto a human 1.7K cDNA microarray (University Health Network Microarray Center, Toronto, Canada). This microarray contains 1,781 double-spotted probes corresponding to well-characterized human ESTs. Most of these spots are associated with a Swiss-Prot ID. A 25-mm×60-mm cover slip (Erie Scientific, Portsmouth, N.H.) was gently placed on top of the sample, and hybridization was performed in an Array IT hybridization cassette at 65° C. overnight. Following hybridization, the microarray slide was washed with wash buffer 1 (2×SSC and 0.1% SDS) at 60° C. for 5 minutes in a glass staining dish, then washed with wash buffer 2 (0.2×SSC) at room temperature. The slide was dried by a brief spin at 1,000×g in a tabletop centrifuge.

The hybridized microarray slides were scanned using a GenePix 4000B scanner (Axon Instruments, Foster City, Calif.), and acquired images were analyzed using GENEPIX PRO 4.1 software. Spots with aberrant morphology or intensities below the threshold for detection were flagged and deleted from the data sets. The mean Cy5/Cy3 ratio for the remaining spots was normalized by the ratio of total intensities between Cy5/Cy3 over the entire array using the Standardization and Normalization of Microarray Data (SNOMAD) web utility, based on the R statistical language (Colantuoni 2002). Normalized intensity data was converted into 1092, and log₂ signal ratios and log₂ mean intensities were calculated. Log₂ ratios were normalized across local log₂ mean intensities using the “loess” function of R. The normalized intensity data for H3-K4Me2 and H3-K9Me2 is set forth in Tables 1 and 2, respectively. Pearson's r value was calculated by averaging the normalized log₂ ratios for each gene between all samples. Scatter plots and Pearson correlations between pairs of groups were performed using GraphPad Prism software (GraphPad Software, San Diego, Calif.), and the resulting Pearson's r values were calculated. Significance Analysis of Microarrays (SAM) (Tusher 2001) was used to identify candidate genes. Delta values were chosen at a false discovery rate (FDR) of 1% on microarrays, and candidate genes were defined as those genes with a 635/532 nm ratio of 2.0 or more over the total average ratio.

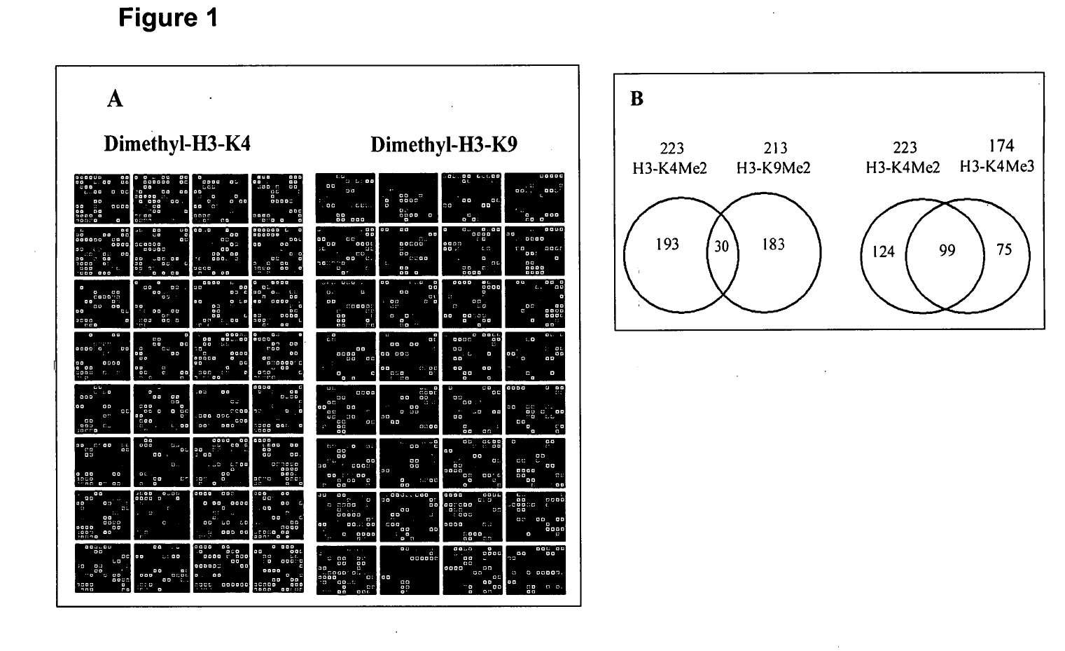

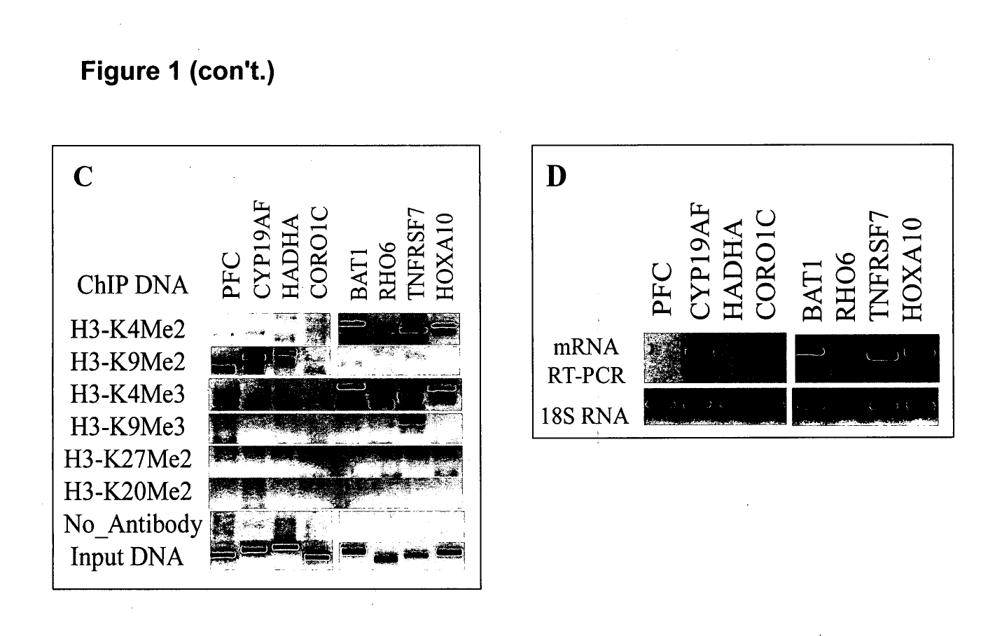

A representative virtual microarray scan is shown in FIG. 1A. Of the 223 H3-K4Me2 candidate genes and 213 H3-K9Me2 candidate genes, only 10 were shared between both groups (FIG. 1B, Table 3). The Pearson correlation-coefficient (r) between H3-K4Me2 and H3-K9Me2 was 0.13 (Table 3). Previous studies analyzing one gene at a time have shown that promoters methylated at H3-K9 are generally not methylated at H3-K4, and that H3-K4 and H3-K9 dimethylation signals represent opposite functions (Noma 2001). The present data suggests that this concept is applicable globally to coding regions represented in the 1.7K cDNA microarray, and is likely applicable genome-wide to human gene coding regions. To verify this, the experiment was repeated using a human 19K-cDNA microarray and a CpG microarray (Universal Health Networks). Results were very similar to those discussed above, with only about 10% of genes exhibiting both H3-K4Me2 and H3-K9Me2.

TABLE 3
Correlation between histone methylation/acetylation at specific lysines in the coding regions of human genes

| | H3- | H3- | H3- | H3- | H3- | H3- | H3- | H4- | H3- |
|---|---|---|---|---|---|---|---|---|---|
| | K9/14Ac | K4Me2 | K4Me3 | K36Me2 | K79Me2 | K9Me2 | K9Me3 | K20Me2 | K27Me2 |
| H3- | 231 | | | | | | | | |
| K9/14Ac | (1) | | | | | | | | |
| H3- | 129 | 223 | | | | | | | |
| K4Me2 | (0.72) | (1) | | | | | | | |
| H3- | 100 | 99 | 174 | | | | | | |
| K4Me3 | (0.64) | (0.58) | (1) | | | | | | |
| H3- | 54 | 24 | 12 | 134 | | | | | |
| K36Me2 | (0.56) | (0.37) | (0.35) | (1) | | | | | |
| H3- | 95 | 63 | 46 | 108 | 179 | | | | |
| K79Me2 | (0.72) | (0.61) | (0.56) | (0.81) | (1) | | | | |
| H3- | 29 | 30 | 30 | 9 | 14 | 213 | | | |
| K9Me2 | (0.12) | (0.13) | (0.09) | (0.01) | (0.04) | (1) | | | |
| H3- | 13 | 18 | 5 | 3 | 5 | 10 | 93 | | |
| K9Me3 | (−0.06) | (−0.08) | (0.03) | (−0.15) | (−0.1) | (0.22) | (1) | | |
| H4- | 9 | 22 | 3 | 2 | 3 | 10 | 52 | 88 | |
| K20Me2 | (−0.21) | (0.09) | (−0.24) | (−0.61) | (−0.39) | (0.03) | (0.54) | (1) | |
| H3- | 16 | 28 | 8 | 1 | 4 | 12 | 62 | 83 | 138 |
| K27Me2 | (−0.28) | (0.001) | (−0.26) | (−0.69) | (−0.49) | (0.06) | (0.48) | (0.92) | (1) |

Top value represents the number of shared genes.
Bottom value represents Pearson correlation coefficient (r)

To further validate the accuracy of the ChIP/cDNA microarray findings, conventional ChIP was performed on a random selection of candidate genes. ChIP primers were designed for four candidate genes from the H3-K9Me2 list (CORO1C, HADHA, PFC, and CYP19AF) and four candidate genes from the H3-K4Me2 list (RHO6, BAT1, HOXA10, and TNFRSF7). The sequences of these primers are set forth in SEQ ID NOs: 4-19. ChIP results confirmed that DNA ChIPed with H3-K9Me2 antibody is enriched with the H3-K9Me2 candidate genes but not with the H3-K4Me2 candidate genes (FIG. 1C). Similarly, DNA ChIPed with H3-K4Me2 antibody is enriched with the H3-K4Me2 candidate genes but not with the H3-K9Me2 candidate genes. These results confirm that the present ChIP/cDNA microarray approach provides a reliable and accurate method for profiling histone modifications.

### Example 2

### RT-PCR Analysis of the Expression of Genes Associated with H3-K4 or H3-K9 Dimethylation

Previous studies suggest that methylation of H3-K9 is generally related to gene silencing or repression, while methylation of H3-K4 is generally considered to be associated with transcriptionally active euchromatin (Sims 2003).

RT-PCR was performed on four candidate genes from the H3-K9Me2 list (CORO1C, HADHA, PFC, and CYP19AF and four candidate genes from the H3-K4Me2 list (RHO6, BAT1, HOXA10, and TNFRSF7) to determine whether dimethylation of H3-K4 or H3-K9 correlates with mRNA expression. The primers used for these experiments are set forth in SEQ ID NOs: 20-35. The four H3-K4Me2 candidate genes were all clearly expressed, while three of the H3-K9Me2 candidate genes (PFC, HADHA, and CORO1C) showed no mRNA expression (FIG. 1D). The fourth H3-K9Me2 candidate gene, CYP19AF, displayed a low level of expression (FIG. 1 D). These results validate the contention that H3-K4Me2 and H3-K9Me2 have opposite functions, namely gene activation versus gene silencing.

### Example 3

### ChIP/Promoter Microarray Analysis of H3-K4 and H3-K9 Dimethylation

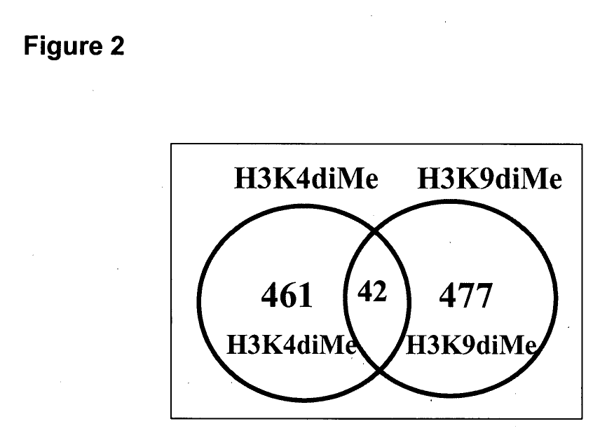

The ChIP/cDNA microarray experiments from Example 1 were repeated, but with a human promoter microarray substituted for the cDNA microarray. The hu6k human promoter microarray (Li, Z. 2003) (Aviva Bioscience, San Diego, Calif.) contains PCR products spanning the proximal promoters of 4,839 human genes. The results of this analysis were very similar to those obtained in Example 1 using the cDNA microarray. Of the 461 H3-K4Me2 candidate genes and 477 H3-K9Me2 candidate genes, only 42 were shared between both groups (FIG. 2). This further reinforces the notion that H3-K4Me2 and H3-K9Me2 play opposite functional roles, namely gene activation versus gene silencing.

### Example 4

### ChIP/cDNA Microarray Analysis of H3-K4 Trimethylation

A unique feature of histone lysine methylation is that it has three levels of methylation states. The ε-amino group of lysine can be mono-, di-, or trimethylated by distinct histone methylases. Although the biological significance of these levels of methylation is unknown, several previous studies have indicated that methylation level correlates with gene expression. For example, trimethylated H3-K4 (H3-K4Me3) is exclusively present in newly active genes, and therefore seems to define an active state of gene expression (Santos-Rosa 2002).

The ChIP/cDNA microarray procedure described in Example 1 was repeated in human THP-1 monocytes using antibodies against H3-K4Me3. The normalized intensity data for H3-K4Me3 is set forth in Table 4. As expected, H3-K4Me3 displayed a marked overlap with H3-K4Me2 (FIG. 1 B). Among 174 H3-K4Me3 candidate genes, 99 were shared with H3-K4Me2 (FIG. 1 B, Table 3). The Pearson correlation-coefficient (r) between H3-K4Me3 and H3-K4Me2 was 0.58 (Table 3). This confirms that H3-K4 di- and trimethylation levels closely correlate with one another in the coding regions of human genes. As suggested earlier (Santos-Rosa 2002), genes displaying H3-K4 trimethylation may represent recently transcribed genes.

### Example 5

### ChIP/cDNA Microarray Analysis of H3-K4, H3-K79, and H3-K36 Dimethylation

Like H3-K4, methylation of H3-K79 and/or H3-K36 is generally considered to be associated with transcriptionally active euchromatin (Sims 2003). In addition, recent reports have demonstrated that methylation of H3-K4 and H3-K79 is associated with active genes in Drosophila (Schubeler 2004).

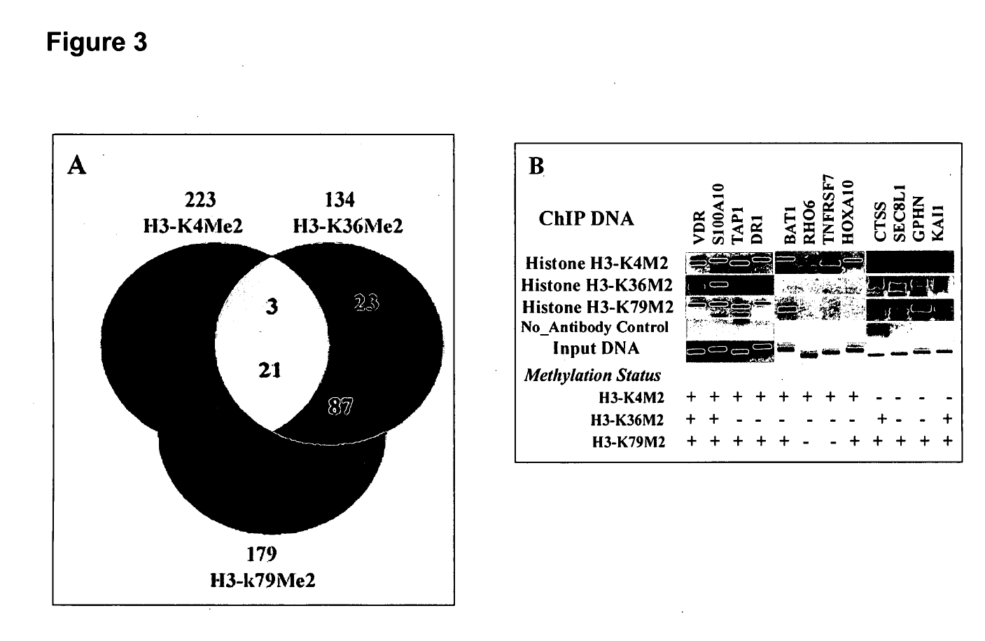

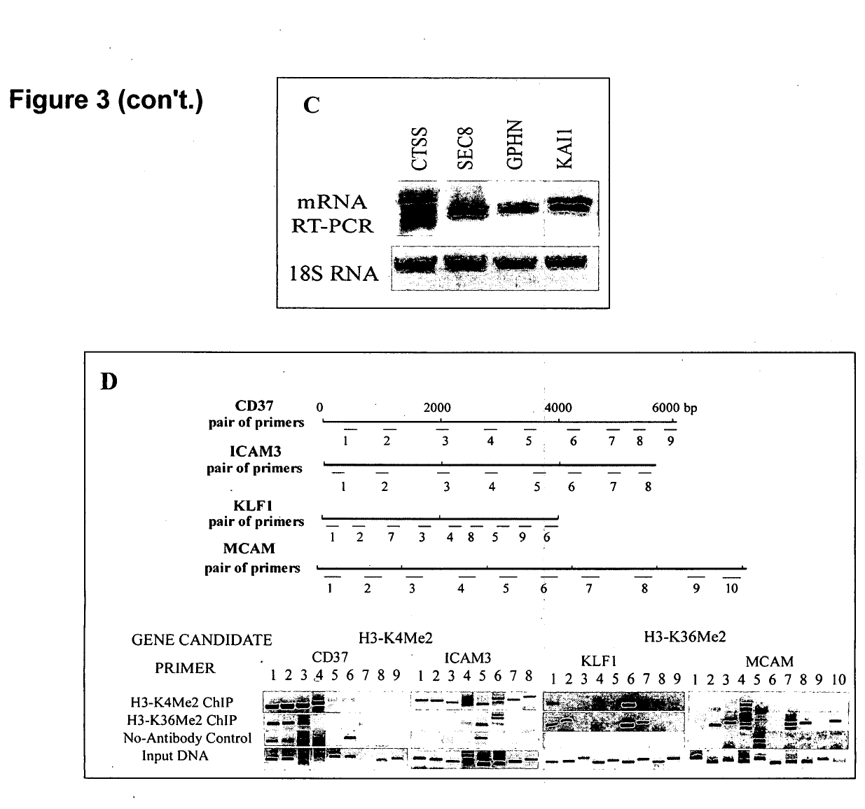

The ChIP/cDNA microarray procedure described in Example 1 was repeated in human THP-1 monocytes using antibodies against H3-K79Me2 and H3-K36Me2. The normalized intensity data for H3-K79Me2 and H3-K36Me2 is set forth in Tables 5 and 6, respectively. These results showed that H3-K79 dimethylation is closely associated with H3-K4 dimethylation (FIG. 3A). Among the 223 H3-K4Me2 and 179 H3-K79Me2 candidate genes, there was an overlap of approximately 63 genes (FIG. 3A, Table 3). The Pearson correlation-coefficient (r) between H3-K4Me2 and H3-K79Me2 was 0.61 (Table 3). This supports the idea that H3-K4Me2/H3-K79Me2 represents a specific methylation pattern in gene coding regions. The results also showed that H3-K36 dimethylation is closely associated with H3-K79 dimethylation. Among the 134 H3-K36Me2 and 179 H3-K79Me2 candidate genes, there was an overlap of approximately 108 genes (FIG. 3A, Table 3). The Pearson correlation-coefficient (r) between H3-K36Me2 and H3-K79Me2 was 0.81 (Table 3). This association represents a newly identified methylation pattern in active coding regions, H3-K36Me2/H3-K79Me2. Interestingly, however, no significant overlap was observed between the H3-K36Me2 and H3-K4Me2 candidate genes. Among the 223 H3-K4Me2 and 134 H3-K36Me2 candidate genes, there was an overlap of only about 24 genes (FIG. 3A, Table 3). The Pearson correlation-coefficient (r) between H3-K4Me2 and H3-K36Me2 was 0.37 (Table 3). Thus, H3-K36Me2 is associated with H3-K79Me2 but not with H3-K4Me2, despite the fact that all three methylation marks are associated active gene coding regions.

In mammals, Dot1L methylases are currently implicated in H3-K79 methylation (Feng 2002). In yeast and humans, H3-K36 is methylated by SET2 and NSD1, respectively (Rayasam 2003). The present data suggests that dual histone methylations occur at the ORFs of transcriptionally active genes in the form of H3-K4Me2/H3-K79Me2 and/or H3-K36Me2/H3-K79Me2, but not in the form of H3-K4Me2/H3-K36Me2. If NSD1 and Dot1L are indeed responsible for methylating H3-K36 and H3-K79 in ORFs, this suggests that they may interact with one another or promote the activity of one another. It is also possible that one or more other enzymes methylate H3-K36 and H3-K79 in human gene coding regions.

To validate the accuracy of the GWLA findings, conventional ChIP was performed on select candidate genes from the H3-K4Me2, H3-K36Me2, and H3-K79Me2 data sets. These select candidate genes, along with the DNA sequences of the ChIP primers for each, were VDR (SEQ ID NOs: 44-45), S100A10 (SEQ ID NOs: 46-47), TAP1 (SEQ ID NOs: 48-49), DR1 (SEQ ID NOs: 50-51), BAT1 (SEQ ID NOs: 14-15), RHO6 (SEQ ID NOs: 12-13), TNFRSF7 (SEQ ID NOs: 18-19), HOXA10 (SEQ ID NOs: 16-17), CTSS (SEQ ID NOs: 52-53), SEC8L1 (SEQ ID NOs: 54-55), GPHN (SEQ ID NOs: 56-57), and KAI1 (SEQ ID NOs: 58-59). The results of the conventional ChIP assay confirmed the microarray data (FIG. 3B). RT-PCR of the four candidate genes CTSS, SEC8L1, GPHN, and KAI1 using the primers set forth in SEQ ID NOs: 3643 verified that all four genes were being expressed (FIG. 3C). Conventional ChIP analysis was then carried out on multiple sections of the transcribed regions of the H3-K4Me2 candidate genes CD37 and ICAM3 and the H3-K36Me2 candidate genes KLF1 and MCAM. The primers used for these experiments are set forth in SEQ ID NOs: 60-131.

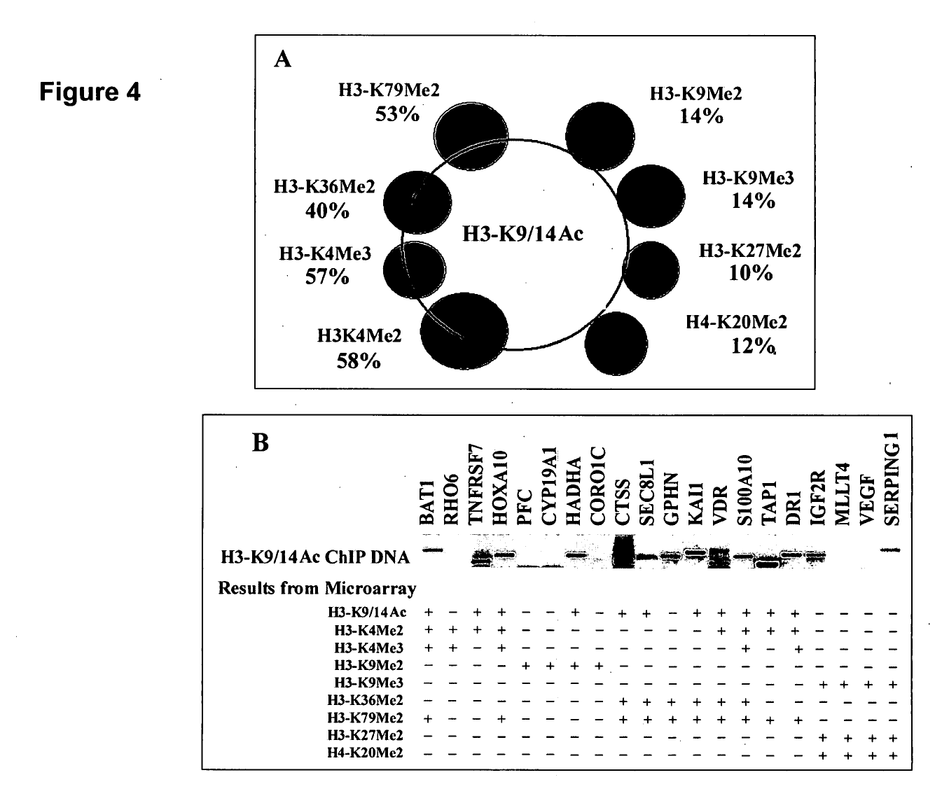

Acetylation levels were measured in candidate genes from various methylation groups, and it was determined that coding regions associated with various methylation markers are hypoacetylated (FIG. 4A). These results were verified by performing conventional ChIP analysis on select candidate genes from the various methylation groups (FIG. 4B). These select candidate genes, along with the DNA sequences of the ChIP primers for each, were BAT1 (SEQ ID NOs: 14-15), RHO6 (SEQ ID NOs: 12-13), TNFRSF7 (SEQ ID NOs: 18-19), HOXA10 (SEQ ID NOs: 16-17), PFC (SEQ ID NOs: 10-11), CYP19A1 (SEQ ID NOs: 8-9), HADHA (SEQ ID NOs: 6-7), CORO1C (SEQ ID NOs: 4-5), CTSS (SEQ ID NOs: 52-53), SEC8L1 (SEQ ID NOs: 54-55), GPHN (SEQ ID NOs: 56-57), KAI1 (SEQ ID NOs: 58-59), VDR (SEQ ID NOs: 44-45), S100A10 (SEQ ID NOs: 46-47), TAP1 (SEQ ID NOs: 48-49), DR1 (SEQ ID NOs: 50-51), IGF2R (SEQ ID NOs: 132-133), MLLT4 (SEQ ID NOs: 134-135), VEGF (SEQ ID NOs: 136-137), and SERPING1 (SEQ ID NOs: 138-139).

### Example 6

### ChIP/cDNA Microarray Analysis of H3-K9 Trimethylation and H4-K20/H3-K27 Dimethylation

Like H3-K9, methylation of H3-K27 and/or H4-K20 is generally related to gene silencing or repression (Sims 2003). However, the global relationship between methylation of H3-K27, H4-K20, and H3-K9 is unknown. In mammals, Suv39h1/Suvh2 has been implicated in trimethylation of H3-K9 (Feng 2002). Likewise, Ezh2, a polycomb group (PcG) protein, and PR-Set7 have been implicated in dimethylation of H3-K27 and H4-K20, respectively (Nishioka 2002).

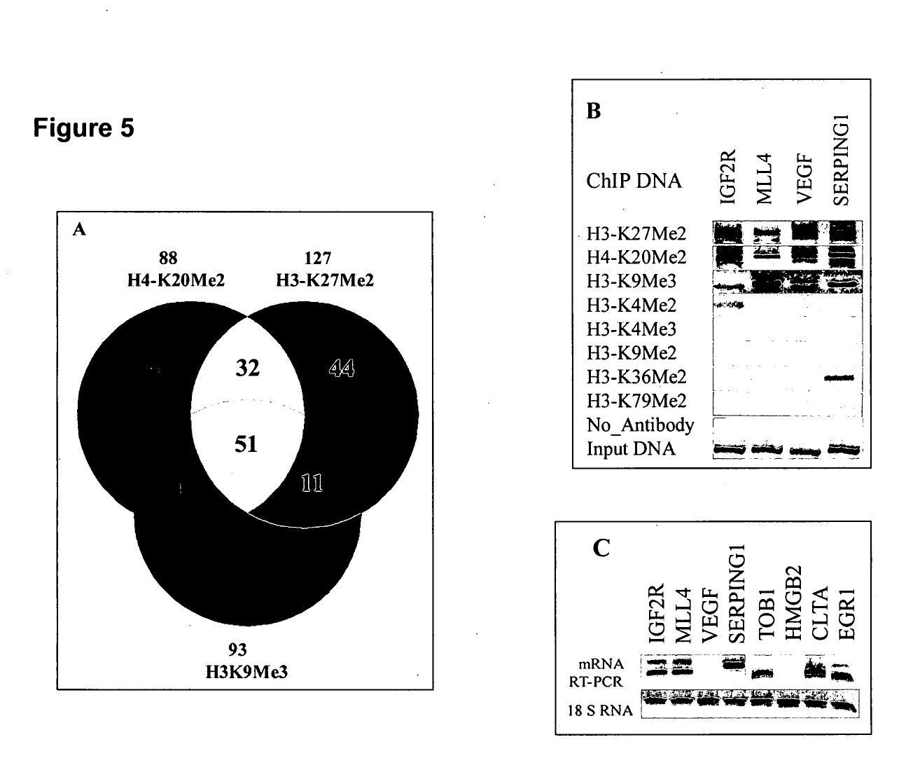

The ChIP/cDNA microarray procedure described in Example 1 was repeated in human THP-1 monocytes using antibodies against H3-K9Me3, H4-K20Me2, and H3-K27Me2. The normalized intensity data for H3-K9Me3, H4-K20Me2, and H3-K27Me2 is set forth in Tables 7, 8, and 9, respectively. These results reveal a close correlation among H3-K9Me3, H4-K20Me2, and H3-K27Me2 in human gene coding regions (FIG. 5A). Among the 88 H4-K20Me2 and 138 H3-K27Me2 candidate genes, there was an overlap of approximately 83 genes (FIG. 5A, Table 3). The Pearson correlation-coefficient (r) between H4-K20Me2 and H3-K27Me2 was 0.92 (Table 3). Among the 88 H4-K20Me2 and 93 H3-K9Me3 candidate genes, there was an overlap of approximately 52 genes (FIG. 5A, Table 3). The Pearson correlation-coefficient (r) between H4-K20Me2 and H3-K9Me3 was 0.54 (Table 3). Among the 127 H3-K27Me2 and 93 H3-K9Me3 candidate genes, there was an overlap of approximately 62 genes (FIG. 5A, Table 3). The Pearson correlation-coefficient (r) between H3-K27Me2 and H3-K9Me3 was 0.48 (Table 3). In addition, 51 candidate genes were shared among H3-K9Me3, H4-K20Me2, and H3-K27Me2. This overlap suggests an H3-K9Me3/H4-K20Me2/H3-K27Me2 methylation pattern occurring in human gene coding regions.

To validate the accuracy of the GWLA findings, conventional ChIP was performed on four select candidate genes present in H3-K9Me3, H4-K20Me2, and H3-K27Me2: IGF2R, MLL4, VEGF, and SERPING1. These results confirmed the microarray data (FIG. 5B). However, RT-PCR of these selected candidate genes, as well as four additional candidate genes (TOB1, HMGB2, CLTA, and EGR1), indicated that they were not necessarily silenced (FIG. 5C). These RT-PCR experiments were carried out using the primers set forth in SEQ ID NOs: 140-155. Out of eight genes tested, six were expressed and two were not expressed. Thus, the consequences of the H3-K9Me3/H4-K20Me2/H3-K27Me2 methylation pattern appear to be different from those of H3-K9Me2 alone, which is an established marker of gene repression or silencing. This difference may be due to heterozygosity of methylation. For example, a monoallelic gene may possess the H3-K9Me3/H4-K20Me2/H3-K27Me2 methylation pattern on only one of two gene copies. In this situation, the gene copy displaying H3-K9Me3/H4-K20Me2/H3-K27Me2 methylation may be repressed, while the other gene copy may be expressed normally.

### Example 7

### ChIP/cDNA Microarray Analysis of H3-K9/K14 Acetylation

Histone acetylation has been genetically and biochemically linked to transcriptional activation (Wu 2000; Okamoto 2004). Thus, it is possible that correlations between histone acetylation and methylation at specific histone lysine residues may reflect the potential transcriptional activity status of the methylated histone.

The ChIP/cDNA microarray procedure described in Example 1 was repeated in human THP-1 monocytes using antibodies against H3-K9Ac and H3-K14Ac. Acetylation at H3-K9/K14 is associated with gene activation. The normalized intensity data for H3-K9/K14Ac, which is set forth in Table 10, was compared to the data obtained in Examples 1, 4, 5, and 6. A close correlation was observed between H3-K9/K14 acetylation and H3-K4, H3-K36, and H3-K79 methylation (FIG. 4, Table 3). H3-K9/Kl4Ac candidate genes overlapped with 129 of the 223 H3-K4Me2 candidate genes (r=0.72), 100 of the 174 H3-K4Me3 candidate genes (r=0.64), 54 of the 134 H3-K36Me2 candidate genes (r=0.56), and 95 of the 179 H3-K79Me2 candidate genes (r=0.72) (FIG. 4, Table 3). This is consistent with the idea that methylation at these sites is associated with active transcription. On the other hand, poor correlation was observed between H3-K9/K14 acetylation and H3-K9, H3-K27, and H4-K20 methylation (FIG. 4, Table 3). H3-K9/K14 acetyl candidate genes overlapped with only 29 of the 213 H3-K9Me2 candidate genes (r=0.12), 13 of the 93 H3-K9Me3 candidate genes (r=−0.06), 9 of the 88 H3-K20Me2 candidate genes (r=-0.21), and 16 of the 138 H4-K27Me2 candidate genes (r=−0.28) (FIG. 4, Table 3). This is consistent with the idea that methylation at these sites is not associated with active transcription.

### Example 8

### ChIP/cDNA Microarray Analysis of H3-K9 Dimethylation Under High Glucose vs. Normal Glucose Conditions

Previous experiments have established that high glucose (HG) conditions increase acetylation of H3-K9 and H3-K14 at the TNF-α promoter in THP-1 cells (Miao 2004). To determine the effects of HG conditions on histone methylation, THP-1 monocytes were grown in either HG (25 mM) or normal glucose (NG) conditions for 72 hours. ChIP was performed as described in Example 1 using antibodies against H3-K9Me2. ChIPed DNA from HG cells was labeled with Cy5-dCTP, while ChIPed DNA from NG cells was labeled with Cy3-dCTP. The samples were combined and hybridized to the human 1.7K cDNA microarray, and Cy5/Cy3 intensity was measured as set forth in Example 1. Genes displaying a Cy5/Cy3 ratio of greater than 2.0 were classified as having increased H3-K9Me2, while genes displaying a Cy5/Cy3 ratio of less than 0.5 were classified as having decreased H3-K9Me2. Approximately 50 genes were identified as falling into one of these two groups, with HG conditions increasing H3-K9Me2 in some genes and decreasing H3-K9Me2 in others. Presumably, those genes with increased H3-K9Me2 are silenced, while those genes with decreased H3-K9Me2 are activated. This data represents a novel view of gene expression under HG or diabetic conditions that differs from mRNA expression data.

### Example 9

### ChIP/cDNA Microarray Analysis of H3-K9 Dimethylation in Normal vs. Type 1 Diabetes Subjects

Previous experiments have established that acetylation of H3-K9 and H3-K14 is increased in subjects with T1D (Miao 2004). To determine whether T1D was associated with similar changes in histone methylation, blood samples were obtained from T1D subjects and matched controls, and monocyte and lymphocyte fractions were prepared from these samples. ChIP was performed as described in Example 1 using antibodies against H3-K9Me2. In each fraction, ChIPed DNA from T1D subjects was labeled with Cy5-dCTP, while ChIPed DNA from normal subjects was labeled with Cy3-dCTP. The samples were combined and hybridized to the human 1.7K cDNA microarray, and Cy5/Cy3 intensity was measured as set forth in Example 1. Genes displaying a Cy5/Cy3 ratio of greater than 2.0 were classified as having increased H3-K9Me2, while genes displaying a Cy5/Cy3 ratio of less than 0.5 were classified as having decreased H3-K9Me2. A partial listing of genes associated with increased and decreased H3-K9Me2 in T1D monocytes is set forth in Tables 11 and 12, respectively. Presumably, those genes with increased H3-K9Me2 are silenced in T1D monocytes versus normal monocytes, while those genes with decreased H3-K9Me2 are activated in T1D monocytes versus normal monocytes. Interestingly, significant differences were observed in the histone methylation patterns of monocytes and lymphocytes in both normal and T1D samples.

TABLE 11
Genes displaying increased H3-K9Me2 in T1D monocytes

| Accession | | |
|---|---|---|
| # | Name | Description |
| AA002125 | BIRC2 | baculoviral IAP repeat-containing 3 |
| AA010480 | CYP2J2 | cytochrome P450, family 2 |
| AA010522 | UTRN | utrophin (homologous to dystrophin) |
| T74462 | TAP1 | transporter 1, ATP-binding cassette |
| AA011445 | CASP3 | caspase 3, apoptosis-related cysteine protease |
| AA029803 | HDLBP | high density lipoprotein binding protein |
| | | (vigilin) |
| AA031420 | TNFRSF6 | tumor necrosis factor receptor superfamily |
| AA034352 | MEF2A | MADS box transcription factor enhancer |
| | | factor 2 |
| AA115108 | CD59 | CD59 antigen p18-20 |
| AA128249 | FABP4 | fatty acid binding protein 4, adipocyte |
| AA151197 | ITGA5 | integrin, alpha 5 (fibronectin receptor) |
| AA203133 | EDG5 | sphingolipid G-protein-coupled receptor, 5 |
| AL521184 | OCRL | oculocerebrorenal syndrome of Lowe |
| AL574986 | CIAO1 | WD40 protein Ciao1 |
| AU131389 | NUP214 | nucleoporin 214 kDa |
| BF918489 | APLP2 | amyloid beta (A4) precursor-like protein 2 |
| BG958992 | SH2D4A | SH2 domain containing 4A |
| BI222736 | P15RS | hypothetical protein FLJ10656 |
| H03156 | GFPT1 | glutamine-fructose-6-phosphate transaminase 1 |
| H04421 | DUSP1 | dual specificity phosphatase 1 |
| H04530 | ECHS1 | enoyl Coenzyme A hydratase |
| H15472 | GRIK2 | glutamate receptor, ionotropic, kainite 2 |
| H17813 | TOP2A | topoisomerase (DNA) II alpha 170 kDa |
| AA040480 | BAT2 | HLA-B associated transcript 2 |
| H23380 | VPS41 | vacuolar protein sorting 41 (yeast) |
| H24707 | DLG1 | discs, large homolog 1 (Drosophila) |

TABLE 12
Genes displaying decreased H3-K9Me2 in T1D monocytes

| Accession | | |
|---|---|---|
| # | Name | Description |
| AA010882 | GYS1 | glycogen synthase 1 (muscle) |
| AA010929 | MPG | N-methylpurine-DNA glycosylase |
| AA056153 | SERPING1 | serine (or cysteine) proteinase inhibitor |
| AA099193 | CLTA | clathrin, light polypeptide (Lca) |
| AA150817 | TXN | thioredoxin |
| AA203405 | HD | huntingtin (Huntington disease) |
| AA203732 | CHP | calclium binding protein P22 |
| AI826395 | PPP2R3A | protein phosphatase 2 (formerly 2A) |
| BE748191 | STMN1 | stathmin 1/oncoprotein 18 |
| BE870715 | IGF2R | insulin-like growth factor 2 receptor |
| BF346332 | EPN2 | epsin 2 |
| BF686093 | GAPD | glyceraldehyde-3-phos dehydrogenase |
| BG166946 | RHEB | Ras homolog enriched in brain |
| BG393106 | FALZ | fetal Alzheimer antigen |
| BG530809 | POLR2G | polymerase (RNA) II polypeptide G |
| BG618848 | SAA2 | serum amyloid A2 |
| BG753663 | TUBB5 | tubulin, beta, 5 |
| BG755562 | CFL1 | cofillin 1 (non-muscle) |
| BG827779 | COBRA1 | cofactor of BRCA1 |
| BI259257 | CCT3 | chaperonin containing TCP1, subunit 3 |
| N25099 | HLA-DPA1 | major histocompatibility complex, class II |
| BM011621 | SLC25A6 | solute carrier family 25, member 6 |
| BM044161 | PRDX6 | peroxiredoxin 6 |
| H08978 | CACNB3 | calcium channel, beta 3 subunit |
| H10106 | MLLT3 | myeloid/lymphoid (trithorax homolog) |
| H17600 | MAP1B | microtubule-associated protein 1B |

Several of the genes exhibiting decreased H3-K9Me2 in T1D monocytes also exhibited decreased H3-K9Me2 in HG cultured THP-1 cells (Example 9). A partial listing of these genes is set forth in Table 13.

TABLE 13
Genes displaying decreased H3-K9Me2 in both
T1D monocytes and HG cultured THP-1 cells

| Accession # | Name |
|---|---|
| R15256 | RDC1 |
| BG753663 | TUBB5 |
| BG530809 | POLR2G |
| R14094 | NCDN |
| BG622891 | PSG5 |
| W47595 | TGFB2 |
| AA011479 | EPHX2 |
| AA015685 | ZNF33A |
| H65733 | KLF1 |
| R22872 | CTSS |
| R85021 | FGFR3 |
| AA056153 | SERPING1 |
| W48791 | VIL2 |
| W95953 | EWSR1 |
| W45582 | LGALS3 |
| W00854 | TRIP-Br2 |
| BG178139 | WBSCR1 |
| W69683 | NFE2L1 |
| R73338 | RUNX1 |
| BG755562 | CFL1 |
| W79101 | AMT |
| AA020894 | MAP2 |
| BI084076 | SLC25A1 |
| AA133258 | HADHB |
| R54267 | BCR |
| R97966 | ACADVL |
| W01850 | POLD2 |
| R07395 | CASP8 |
| BM044542 | CDK4 |
| T77120 | STAR |
| BG029087 | PABPC1 |
| R47859 | NPR1 |
| H29950 | TOB1 |
| T77500 | MAPK4 |
| N40420 | CCND1 |
| W25385 | PHKA2 |
| W52156 | OXTR |
| R14146 | GSTM5 |
| AA017038 | SNAP25 |
| BE870715 | IGF2R |
| H75902 | CR1 |
| N25099 | HLA-DPA1 |
| BM006611 | KRTHB1 |
| BF218768 | CDC10 |
| N53295 | RPL39L |
| W31016 | IL6 |
| AA042836 | IRF2 |
| BM011621 | SLC25A6 |

### Example 10

### Identification of Genomic “Hotspots” Affected by HG or T1D

Several of the genes that displayed altered H3-K9Me2 patterns in both T1D monocytes and HG cultured THP-1 cells in.Example 9 were located in genomic “hotspots.” For example, BAT1, BAT2, TAP1, HLA-DPA1, BAT8, and HSPA1A are all located within the key MHC locus (6p21.3), which is strongly associated with T1D. BAT1, BAT2, and TAP1 all displayed increased H3-K9Me2 in T1D and HG cells, while HLA-DPA1, BAT8, and HSPA1A all displayed decreased H3-K9Me2 in T1D and HG cells. These findings suggest that the expression of genes in particular chromosomal regions may be sensitive to HG and T1D.

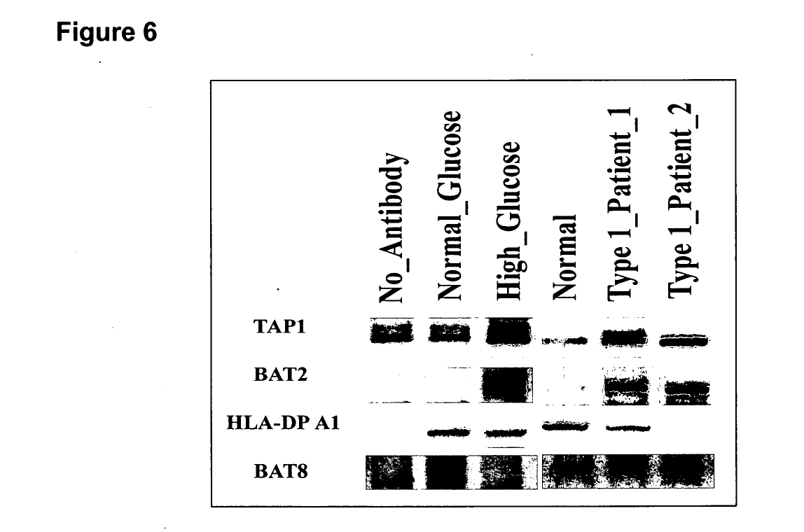

Follow-up ChIP assays were performed on four of the genes located in the chromosome 6p21.3 “hotspot”: TAP1, BAT2, HLA-DPA1, and BAT8. Each of these genes is related to the pathology of T1D. The assays confirmed the results obtained in Example 9. TAP1 and BAT2 displayed increased H3-K9Me2 in HG and T1D monocytes, while HLA-DPA1 and BAT8 displayed decreased H3-K9Me2 (FIG. 6).

### Example 11

### ChIP/Promoter Microarray Analysis of H3-K9Me2 in Normal vs. Type 1 Diabetes Subjects

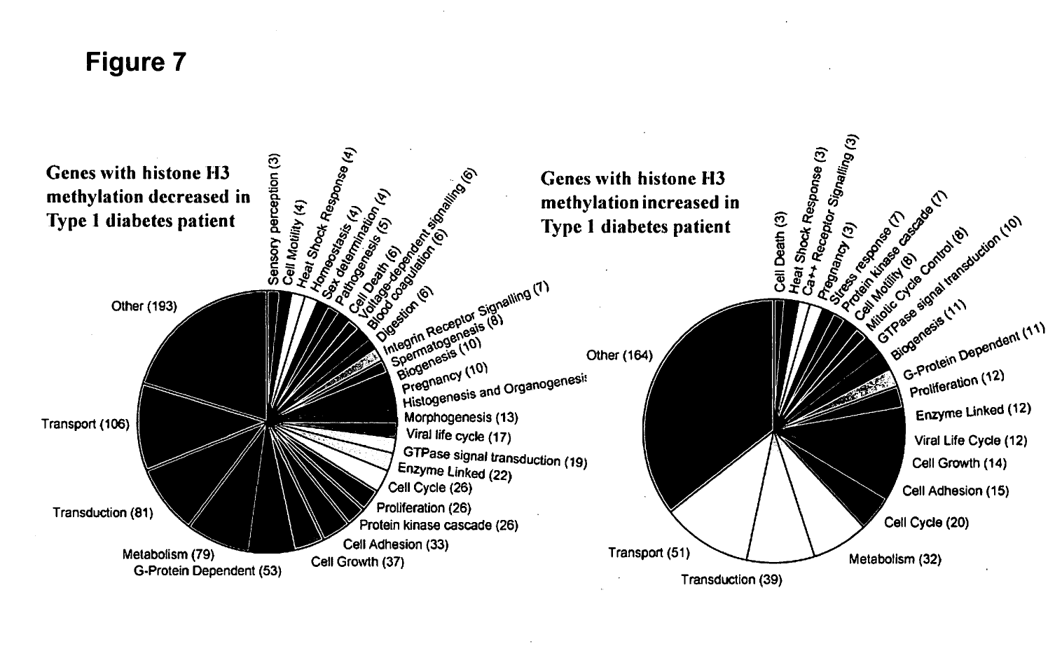

The ChIP/cDNA microarray experiments from Example 9 were repeated, but with the hu6k promoter microarray substituted for the cDNA microarray. Sets of genes were identified that were associated with both increased and decreased H3-K9Me2 in T1D monocytes versus normal monocytes (FIG. 7).

### Example 12

### ChIP/Microarray Analysis of H3-K9Me2 and H3-K4Me2 in IT vs. CT T1D Subjects

Blood samples will be obtained from IT and CT T1D subjects, and monocytes and lymphocytes will be isolated as described previously (Shanmugam 2003b; Miao 2004; Shanmugam 2004). The ChIP/cDNA microarray procedure described in Example 1 will be carried out on these cells using antibodies against H3-K9Me2, H3-K4Me2, and H3-K27Me2. Microarrays employed for these experiments will include regular cDNA microarrays, promoter microarrays, and the PancChip (Scearce 2002), which contains probes from a highly focused set of diabetes-related human genes. This procedure will identify differential histone methylation patterns between CT and IT subjects in key genes associated with autoimmunity, inflammation, and various diabetic complications. CT and IT samples may be further divided into groups according to the severity of various T1D-associated microvascular complications exhibited by the subjects from which they are obtained. This will allow for the identification of histone methylation patterns associated with different stages and severities of T1D.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Bannister, A. J., Schneider, R., Kouzarides, T. 2002. Histone methylation: dynamic or static? Cell 109:801-806.
2. Baynes, J. W. 1991. Role of oxidative stress in development of complications in diabetes. Diabetes 40:405-412.
3. Beisel, C., et al. 2002. Histone methylation by the Drosophila epigenetic transcriptional regulator Ash1. Nature 419:857-862.
4. Berger, S. L. 2002. Histone modifications in transcriptional regulation. Curr Opin Genet Dev 12:142-148.
5. Bernstein, B. E., et al. 2002. Methylation of histone H3 Lys 4 in coding regions of active genes. Proc Natl Acad Sci USA 99:8695-8700.
6. Brownlee, M. Cerami, A., Vlassara, H. 1988. Advanced glycosylation end products in tissue and the biochemical basis of diabetic complications. N Engl J Med 318:1315-1321.
7. Cao, R., Wang, L., Wang, L., Xia, H. 2002. Role of histone H3 lysine 27 methylation in Polycomb-group silencing. Science 298:1039-1043.
8. Cipollone, F., et al. 2003. The receptor RAGE as a progression factor amplifying arachidonate-dependent inflammatory and proteolytic response in human atherosclerotic plaques: role of glycemic control. Circulation 108:1070-1077.
9. Colantuoni, C., Henry, G., Zeger, S., Pevsner, J. 2002. SNOMAD (Standardization and Normalization of MicroArray Data): web-accessible gene expression data analysis. Bioinformatics 18:1540-1541.
10. Czermin, B., et al. 2002. Drosophila enhancer of Zeste/ESC complexes have a histone H3 methyltransferase activity that marks chromosomal Polycomb sites. Cell 111:185-196.
11. Dover, J. J., et al. 2002. Methylation of histone H3 by COMPASS requires ubiquitination of histone H2B by Rad6. J Biol Chem 277:28368-28371.
12. Egger, G., Liang, G., Aparicio, A., Jones, P. A. 2004. Epigenetics in human disease and prospects for epigenetic therapy. Nature 429:457-463.
13. Feng, Q., et al. 2002. Methylation of H3-lysine 79 is mediated by a new family of HMTases without a SET domain. Curr Biol 12:1052-1058.
14. Florez, J. C., Hirschhorn, J., Altshuler, D. 2003. The inherited basis of diabetes mellitus: implications for the genetic analysis of complex traits. Annu Rev Genomics Hum Genet 4:257-291.
15. Gerber, M., Shilatifard, A. 2003. Transcriptional elongation by RNA polymerase II and histone methylation. J Biol Chem 278:26303-26306.
16. Goo, Y. H., et al. 2003. Activating signal cointegrator 2 belongs to a novel steady-state complex that contains a subset of trithorax group proteins. Mol Cell Biol 23:140-149.
17. Guha, M., Bai, W., Nadler, J., Natarajan, R. 2000. Molecular mechanisms of TNF-alpha gene expression in monocytic cells via hyperglycemia-induced oxidant stress dependent and independent pathways. J Biol Chem 275:17728-17739.
18. Hampsey, M., Reinberg, D. 2003. Tails of intrigue: phosphorylation of RNA polymerase II mediates histone methylation. Cell 113:429-432.
19. Hofmann, M. A., et al. 1998. Insufficient glycemic control increases nuclear factor-kappa B binding activity in peripheral blood mononuclear cells isolated from patients with type 1 diabetes. Diabetes Care 21:1310-1316.
20. Ido, Y., Kilo, C., Williamson, J. R. 1997. Cytosolic NADH/NAD+, free radicals, and vascular dysfunction in early diabetes mellitus. Diabetologia 40 Suppl 2:S115-S117.
21. Iizuka, M., Smith, M. M. 2003. Functional consequences of histone modifications. Curr Opin Genet Dev 13:154-160.
22. Ishii, H., Daisuke, K., King, G. L. 1998. Protein kinase C activation and its role in the development of vascular complications in diabetes mellitus. J Mol Med 76:21-31.
23. Jain, S. K., et al. 2002. Hyperketonemia increases tumor necrosis factor-alpha secretion in cultured U937 monocytes and Type 1 diabetic patients and is apparently mediated by oxidative stress and cAMP deficiency. Diabetes 51:2287-2293.
24. Jenuwein, T., Allis, C. D. 2001. Translating the histone code. Science 293:1074-1079.
25. Kirmizis, A., et al. 2004. Silencing of human polycomb target genes is associated with methylation of histone H3 Lys 27. Genes Dev 18:1592-1605.
26. Krogan, N. J., et al. 2003a. The Paf1 complex is required for histone H3 methylation by COMPASS and Dot1p: linking transcriptional elongation to histone methylation. Mol Cell 11:721-729.
27. Krogan, N. J., et al. 2003b. Methylation of histone H3 by Set2 in Saccharomyces cerevisiae is linked to transcriptional elongation by RNA polymerase 11. Mol Cell Biol 23:4207-4218.
28. Kuzmichev, A., et al. 2002. Histone methyltransferase activity associated with a human multiprotein complex containing the Enhancer of Zeste protein. Genes Dev 16:2893-2905.
29. Lachner, M., et al. 2001. Methylation of histone H3 lysine 9 creates a binding site for HP1 proteins. Nature 410:116-120.
30. Lachner, M., Jenuwein, T. 2002. The many faces of histone lysine methylation. Curr Opin Cell Biol 14:286-298.
31. Lachner, M., O'Sullivan, R. J., Jenuwein, T. 2003. An epigenetic road map for histone lysine methylation. J Cell Sci 116:2117-2124.
32. Li, B., et al. 2003. The Set2 histone methyltransferase functions through the phosphorylated carboxyl-terminal domain of RNA polymerase II. J Biol Chem 278:8897-8903.
33. Li, Z., et al. 2003. A global transcriptional regulatory role for c-Myc in Burkitt's lymphoma cells. Proc Natl Acad Sci USA 100:8164-8169.
34. Litt, M. D., et al. 2001. Correlation between histone lysine methylation and developmental changes at the chicken beta-globin locus. Science 293:2453-2455.
35. Luger, K., et al. 1997. Crystal structure of the nucleosome core particle at 2.8 A resolution. Nature 389:251-260.
36. Ma, H., et al. 2001. Hormone-dependent, CARM1-directed, arginine-specific methylation of histone H3 on a steroid-regulated promoter. Curr Biol 11:1981-1985.
37. Martone, R., et al. 2003. Distribution of NF-kappaB-binding sites across human chromosome 22. Proc Natl Acad Sci USA 100:12247-12252.
38. Miao, F., Lanting, L., Natarajan, R. 2004. In vivo chromatin remodeling events leading to inflammatory gene transcription under diabetic conditions. J Biol Chem 279:18091-18097.
39. Morohoshi, M., Fujisawa, K., Uchimura, I., Numano, F. 1995. The effect of glucose and advanced glycosylation end products on IL-6 production by human monocytes. Ann NY Acad Sci 748:562-570.
40. Muller, J., et al. 2002. Histone methyltransferase activity of a Drosophila Polycomb group repressor complex. Cell 111:197-208.
41. Ng, H. H., Robert, F., Young, R. A., Struhl, K. 2002. Genome-wide location and regulated recruitment of the RSC nucleosome-remodeling complex. Genes Dev 16:806-819.
42. Ng, H. H., Robert, F., Young, R. A., Struhl, K. 2003. Targeted recruitment of Set1 histone methylase by elongating Pol II provides a localized mark and memory of recent transcriptional activity. Mol Cell 11:709-719.
43. Nishikawa, T., et al. 2000. Normalizing mitochondrial superoxide production blocks three pathways of hyperglycemic damage. Nature 404:787-790.
44. Nishioka, K., et al. 2002. PR-Set7 is a nucleosome-specific methyltransferase that modifies lysine 20 of histone H4 and is associated with silent chromatin. Mol Cell 9:1201-1213.
45. Noma, K., Allis, C. D., Grewal, S. I. 2001. Transitions in distinct histone H3 methylation patterns at the heterochromatin domain boundaries. Science 293:1150-1155.
46. Odom, D. T., et al. 2004. Control of pancreas and liver gene expression by HNF transcription factors. Science 303:1378-1381.
47. Okamoto, I., et al. 2004. Epigenetic dynamics of imprinted X inactivation during early mouse development. Science 303:644-649.
48. Orlando, V. 2003. Polycomb, epigenomes, and control of cell identity. Cell 112:599-606.
49. Plath, K., et al. 2003. Role of histone H3 lysine 27 methylation in X inactivation. Science 300:131-135.
50. Pollack, J. R., et al. 1999. Genome-wide analysis of DNA copy-number changes using cDNA microarrays. Nat Genet 23:4146.
51. Pugliese, G., Tilton, R. G., Williamson, J. R. 1991. Glucose-induced metabolic imbalances in the pathogenesis of diabetic vascular disease. Diabetes Metab Rev 7:35-59.
52. Rayasam, G. V., et al. 2003. NSD1 is essential for early post-implantation development and has a catalytically active SET domain. Embo J 22:3153-3163.
53. Rea, S., et al. 2000. Regulation of chromatin structure by site-specific histone H3 methyltransferases. Nature 406:593-599.
54. Ren, B., et al. 2000. Genome-wide location and function of DNA binding proteins. Science 290:2306-2309.
55. Rice, J. C., et al. 2003. Histone methyltransferases direct different degrees of methylation to define distinct chromatin domains. Mol Cell 12:1591-1598.
56. Robyr, D., et al. 2002. Microarray deacetylation maps determine genome-wide functions for yeast histone deacetylases. Cell 109:437-446.
57. Roth, S. Y., Denu, J. M., Allis, C. D. 2001. Histone acetyltransferases. Annu Rev Biochem 70:81-120.
58. Ruderman, N., Williamson, J. R., Brownlee, M. 1992. Glucose and diabetic vascular disease. FASEB J 6:2905-2914.
59. Saccani, S., Natoli, G. 2002. Dynamic changes in histone H3 Lys 9 methylation occurring at tightly regulated inducible inflammatory genes. Genes Dev 16:2219-2224.
60. Santos-Rosa, H., et al. 2002. Active genes are tri-methylated at K4 of histone H3. Nature 419:407-411.
61. Scearce, L. M., et al. 2002. Functional genomics of the endocrine pancreas: the pancreas clone set and PancChip, new resources for diabetes research. Diabetes 51:1997-2004.
62. Schmidt, A. M., et al. 1994. Cellular receptors for advanced glycation end products. Implications for induction of oxidant stress and cellular dysfunction in the pathogenesis of vascular lesions. Arterioscler Thromb 14:1521-1528.
63. Schotta, G., et al. 2004. A silencing pathway to induce H3-K9 and H4-K20 trimethylation at constitutive heterochromatin. Genes Dev 18:1251-1262.
64. Schubeler, D., et al. 2004. The histone modification pattern of active genes revealed through genome-wide chromatin analysis of a higher eukaryote. Genes Dev 18:1263-1271.
65. Shanmugam, N., Reddy, M. A., Guha, M., Natarajan, R. 2003a. High glucose-induced expression of proinflammatory cytokine and chemokine genes in monocytic cells. Diabetes 52:1256-1264.
66. Shanmugam, N., Kim, Y. S., Lanting, L., Natarajan, R. 2003b. Regulation of cyclooxygenase-2 expression in monocytes by ligation of the receptor for advanced glycation end products. J Biol Chem 278:34834-34844.
67. Shanmugam, N., Gaw Gonzalo, I., Natarajan, R. 2004. Molecular mechanisms of high glucose-induced cyclooxygenase-2 expression in monocytes. Diabetes 53:795-802.
68. Shilatifard, A. 2004. Transcriptional elongation control by RNA polymerase II: a new frontier. Biochim Biophys Acta 1677:79-86.
69. Silva, J., et al. 2003. Establishment of histone H3 methylation on the inactive X chromosome requires transient recruitment of Eed-Enx1 polycomb group complexes. Dev Cell 4:481-495.
70. Sims, R. J. 3^{rd}, Nishioka, K., Reinberg, D. 2003. Histone lysine methylation: a signature for chromatin function. Trends Genet 19:629-639.
71. Solomon, M. J., Varshavasky, A. 1985. Formaldedyde-mediated DNA-protein crosslinking: a probe for in vivo chromatin structures. Proc Natl Acad Sci USA 82:6470-6474.
72. Strahl, B. D., Allis, C. D. 2000. The language of covalent histone modifications. Nature 403:4145.
73. Sun, Z. W., Allis, C. D. 2002. Ubiquitination of histone H2B regulates H3 methylation and gene silencing in yeast. Nature 418:104-108.
74. Tachibana, M., et al. 2002. G9a histone methyltransferase plays a dominant role in euchromatic histone H3 lysine 9 methylation and is essential for early embryogenesis. Genes Dev 16:1779-1791.
75. Tamaru, H., et al. 2003. Trimethylated lysine 9 of histone H3 is a mark for DNA methylation in Neurosporacrassa. Nat Genet 34:75-79.
76. Tusher, V. G., Tibshirani, R., Chu, G. 2001. Significance analysis of microarrays applied to the ionizing radiation response. Proc Natl Acad Sci USA 98:5116-5121.
77. Turner, B. M. 2002. Cellular memory and the histone code. Cell 111:285-291.
78. van Leeuwen, F., Gottschling, D. E. 2002a. Genome-wide histone modifications: gaining specificity by preventing promiscuity. Curr Opin Cell Biol 14:756-762.
79. van Leeuwen, F., Gafken, P. R., Gottschling, D. E. 2002b. Dot1p modulates silencing in yeast by methylation of the nucleosome core. Cell 109:745-756.
80. Wang, H., et al. 2001. Purification and functional characterization of a histone H3-lysine 4-specific methyltransferase. Mol Cell 8:1207-1217.
81. Weinmann, A. S., Farnham, P. 2002. Identification of unknown target genes of human transcription factors using chromatin immunoprecipitation. Methods 26:37-47.
82. Wu, J., Grunstein, M. 2000. 25 years after the nucleosome model: chromatin modifications. Trends Biochem Sci 25:619-623.
83. Zhang, Y., Reinberg, D. 2001. Transcription regulation by histone methylation: interplay between different covalent modifications of the core histone tails. Genes Dev 15:2343-2360.

What is claimed is:

1. A non-invasive method for mapping histone modifications in a subject comprising:
a) obtaining a blood sample from a subject;
b) performing a chromatin immunoprecipitation assay on said blood sample using one or more antibodies that bind to one or more specific histone modifications, whereby regions of DNA associated with said specific histone modifications are isolated; and
c) identifying the genomic location of said regions of DNA using a microarray.

2. The method of claim 1, wherein said histone modification is a methylation.

3. The method of claim 1, wherein said microarray is a cDNA microarray.

4. The method of claim 3, wherein said histone modifications are located in gene coding regions.

5. A non-invasive method for mapping histone modifications in a subject comprising:
a) obtaining a blood sample from a subject;
b) dividing said blood sample into a chromatin immunoprecipitation sample and a control sample;
c) performing a chromatin immunoprecipitation assay on said chromatin immunoprecipitation sample using an antibody that binds to a specific histone modification, whereby regions of DNA associated with said specific histone modification are isolated;
d) radiolabeling said regions of DNA with a first radiolabel;
e) radiolabeling DNA from said control sample with a second radiolabel;
f) combining said chromatin immunoprecipitation sample and said control sample into a combined sample, and applying the combined sample to a human cDNA microarray;
g) calculating a label ratio for each probe on said cDNA microarray, wherein said label ratio is the ratio of the intensity of said first radiolabel to the intensity of said second radiolabel; and
h) identifying a set of one or more candidate genes associated with said histone modifications, wherein said candidate genes are genes represented on said cDNA microarray by one or more probes displaying a label ratio greater than or equal to a cut-off label ratio.

6. The method of claim 5, wherein said first and second radiolabel are selected from the group consisting of Cy5-dCTP and Cy3-dCTP.

7. The method of claim 5, wherein said cut-off label ratio is 2.0.

8. The method of claim 5, wherein said histone modification is methylation.

9. The method of claim 8, wherein said methylation occurs at H3-K4, H3-K9, H4-K20, H3-K27, H3-K36, or H3-K79.

10. A non-invasive method for diagnosing type 1 diabetes in a subject comprising:
a) obtaining a test blood sample from a subject;
b) obtaining a control blood sample from a known healthy subject;
c) performing a chromatin immunoprecipitation assay on said test blood sample and said control blood sample using an antibody that binds to dimethylated H3-K9 (H3-K9Me2), whereby regions of DNA associated with H3-K9Me2 are isolated;
d) labeling said regions of DNA from said test sample with a first radiolabel;
e) labeling said regions of DNA from said control sample with a second radiolabel;
f) combining said test sample and said control sample into a combined sample;
g) applying said combined sample to a microarray comprising probes from the coding regions of one or more of the human genes set forth in Table 11 and Table 12;
h) calculating a label ratio for said one or more human genes, wherein said label ratio is the ratio of the intensity of said first radiolabel to the intensity of said second radiolabel;
wherein a label ratio of 2.0 or greater or 0.5 or lesser for one or more of said human genes indicates a diagnosis of type 1 diabetes.

11. A non-invasive method for determining whether a subject is at risk for developing type 1 diabetes comprising:
a) obtaining a test blood sample from a subject;
b) obtaining a control blood sample from a known healthy subject;
c) performing a chromatin immunoprecipitation assay on said test blood sample and said control blood sample using an antibody that binds to dimethylated H3-K9 (H3-K9Me2), whereby regions of DNA associated with H3-K9Me2 are isolated;
d) labeling said regions of DNA from said test sample with a first radiolabel;
e) labeling said regions of DNA from said control sample with a second radiolabel;
f) combining said test sample and said control sample into a combined sample;
g) applying said combined sample to a microarray comprising probes from the coding regions of one or more of the human genes set forth in Table 11 and Table 12;
h) calculating a label ratio for said one or more human genes, wherein said label ratio is the ratio of the intensity of said first radiolabel to the intensity of said second radiolabel;
wherein a label ratio of 2.0 or greater or 0.5 or lesser for one or more of said human genes indicates that said subject is at risk for developing type 1 diabetes.

12. A non-invasive method for predicting the accelerated development of vascular complications in a subject with type 1 diabetes comprising:
a) obtaining a test blood sample from a subject;
b) obtaining a control blood sample from a known healthy subject;
c) performing a chromatin immunoprecipitation assay on said test blood sample and said control blood sample using an antibody that binds to dimethylated H3-K9 (H3-K9Me2), whereby regions of DNA associated with H3-K9Me2 are isolated;
d) labeling said regions of DNA from said test sample with a first radiolabel;
e) labeling said regions of DNA from said control sample with a second radiolabel;
f) combining said test sample and said control sample into a combined sample;
g) applying said combined sample to a microarray comprising probes from the coding regions of one or more of the human genes set forth in Table 11 and Table 12;
h) calculating a label ratio for said one or more human genes, wherein said label ratio is the ratio of the intensity of said first radiolabel to the intensity of said second radiolabel;
wherein a label ratio of 2.0 or greater or 0.5 or lesser for one or more of said human genes indicates that said subject is at risk for accelerated development of vascular complications.
